(19) United States
(12) Patent Application Publication
(10) Pub. No.: US 2016/0272694 A1
(43) Pub. Date: Sep. 22, 2016

(54) COMPOSITIONS AND METHODS FOR THE TREATMENT OF ECTODERMAL DYSPLASIA

(71) Applicant: EDIMER PHARMACEUTICALS, INC., Cambridge, MA (US)

(72) Inventors: Kenneth M. Huttner, Chestnut Hill, MA (US); Neil Kirby, Jr., Andover, MA (US); Pascal Schneider, Epalinges (CH); Olivier Gaide, Geneva (CH)

(21) Appl. No.: 14/442,324

(22) PCT Filed: Nov. 13, 2013
(86) PCT No.: PCT/US13/69799

§ 371 Date: May 12, 2015

Related U.S. Application Data
(60) Provisional application No. 61/726,252, filed on Nov. 14, 2012.
(60) Provisional application No. 61/825,227, filed on May 20, 2013.

(51) Int. Cl. C07K 14/705

(52) U.S. Cl. CPC C07K 14/70575; C07K 2319/30

(57) ABSTRACT

The invention relates to pharmaceutical compositions and methods for the treatment of ectodermal displasias via the administration of EDA agonists, in particular EDI200. Use of the compositions and methods described allow for therapeutic dosing and administration regimens in human patients to correct or alter abnormal phenotypes associated with genetic disorders, in particular, XLHED.

### CROSS REFERENCE TO RELATED APPLICATIONS

This application claims priority to U.S. Provisional Application Ser. No. 61/825,227 filed May 20, 2013 and U.S. Provisional Application Ser. No. 61/726,252 filed Nov. 14, 2012, the contents of each of which are incorporated herein by reference in their entirety.

### REFERENCE TO SEQUENCE LISTING

The present application is being filed along with a Sequence Listing in electronic format. The Sequence Listing is provided as a file entitled 20031002PCTSEQLST.txt, created on Nov. 5, 2013, which is 13,681 bytes in size. The information in the electronic format of the sequence listing is incorporated herein by reference in its entirety.

### FIELD OF THE INVENTION

The invention relates to pharmaceutical compounds, compositions, combinations and formulations, methods, assays and kits for treating, correcting, altering, mitigating and/or modifying one or more phenotypic presentations of ectodermal dysplasia in an individual diagnosed with or suffering from XLHED.

### BACKGROUND OF THE INVENTION

X-linked hypohidrotic ectodermal dysplasia (XLHED) is a rare X chromosome-linked genetic disorder. It is the most common of the ectodermal dysplasias, a spectrum of more than 170 genetic disorders that are characterized by at least one primary morphological defect of ectodermal structures (Pinheiro, M. et al., Am J Med Genet. 1994 Nov. 1; 53(2):153-62). Ectodermal embryogenesis contributes to development of the epidermis and associated structures such as sweat glands, sebaceous glands, mammary glands, Meibomian glands, hair follicles and nails. Ectoderm derivatives also include the anterior ⅔ of the oral cavity, and structures including the epithelia of salivary glands, the enamel of teeth, the covering of the tongue, and part of the pituitary gland. XLHED is clinically characterized by fine, sparse hair (hypotrichosis); few and often pointed teeth (marked oligodontia); diminished or absent eccrine function (hypohidrosis) associated with an elevated risk for life-threatening hyperthermia; and a predisposition to serious, clinically-significant respiratory infections associated with reduced secretory gland function. In addition to humans, the disease has been identified in dogs, mice and cattle.

XLHED is caused by mutations in the EDA gene, chromosomal locus XqI2.q13.1 (Kere, J. et al., Nat Genet. 1996 August; 13(4):409-16). The EDA gene encodes several splice variants, the longest of which encodes the 391 a.a. protein EDA-A1 that is a member of the TNF family and binds specifically to its cognate receptor EDAR. Replacement studies in mice and dogs have confirmed that EDA-A1 is the only EDA gene product necessary to activate the EDA/EDAR signaling pathway (Casal, M. L. et al., Am J Hum Genet. 2007 November; 81(5):1050-6; Gaide, O. et al., Nat Med. 2003 May; 9(5):614-8).

The EDA-A1/EDAR pair signals through an adaptor molecule called the ectodysplasin-A receptor associated death domain (EDARADD) and the transcription factor nuclear factor-kappa B (NF-κB) pathway (Elomaa, O. et al., Hum Mol Genet. 2001 Apr. 15; 10(9):953-62; Headon, D. J. et al., Nature. 2001 Dec. 20-27; 414(6866):913-6; Kumar, A. et al., J Biol Chem. 2001 Jan. 26; 276(4):2668-77; Schmidt-Ullrich R, Tobin D J, Lenhard D, Schneider P, Paus R, Scheiderheit C (2006), Development 133: 1045-1057). The interaction of EDA-A1 and EDAR exerts a regulatory role that is tightly associated with epithelial-mesenchymal interactions and pathways that regulate ectodermal appendage formation and organogenesis in the embryo (Laurikkala, J. et al., Dev Biol. 2001 Jan. 15; 229(2):443-55).

Therefore the genotypic incapacity to synthesize functional EDA-A1 protein results in an XLHED phenotype due to defective ectodermal development. EDA-A1 has been shown to be involved in the morphogenesis of hair follicles and tooth buds during early development. The phenotype associated with dysfunctional EDA-A1 is characterized by sparse or absent hair, missing and/or malformed teeth, hypoplastic eccrine glands, recurrent benign infections, and increased susceptibility to bronchitis and pneumonia (Reed, W. B. et al., Arch Dermatol. 1970 August; 102(2):134-43.; Nordgarden, H. et al., Oral Dis. 2001 May; 7(3):163-70). There is significant morbidity and mortality in affected children due to hyperthermia, caused by the inability to sweat. Significant morbidities include increased risk of respiratory tract infections, ocular disease due to dry eyes, as well as difficulties with mastication, growth retardation, poor appearance, and speech impairment resulting from tooth abnormalities (delayed dentition, conical tooth crowns (peg-shaped teeth) and oligodontia). As XLHED is an X chromosome-linked genetic disorder, the clinical phenotype is consistently severe in affected males and more variable in heterozygous females as the result of random X chromosome inactivation.

The first model of XLHED was identified in mice selected from the Black 6 strain for large size which resulted in the spontaneous appearance of a sub-strain with abnormal hair and tooth development. The affected animals (designated “Tabby mice” due to the resemblance of the fur patterning of the heterozygote females to that of the tabby cat) lack functional EDA protein due to a frame-shift mutation resulting in the absence of the domain necessary for receptor binding and signaling that is critical for normal tooth, hair and sweat gland morphogenesis (Ferguson, B. M. et al., Hum Mol Genet. 1997 September; 6(9):1589-94; Srivastava, A. K. et al., Proc Natl Acad Sci USA. 1997 Nov. 25; 94(24):13069-74). Consequently, these mice have no sweat glands and no hair on the tail. The Tabby mouse currently is a widely used model for XLHED.

There is a dog model of the disease that has been used in XLHED studies. A German shepherd puppy was identified with a phenotype similar to human XLHED (Casal, M. L. et al., Mamm Genome. 2005 July; 16(7):524-31), and the effect was later bred into the Beagle strain, which is more commonly used for laboratory experimentation. Beagles carrying the EDA mutation exhibit a phenotype equivalent in many significant respects to that of humans. Advantages of the canine model include high geno-/pheno-copy and a close similarity to human developmental maturation at birth, while disadvantages include the minimal transplacental immunoglobulin transport.

In summary, XLHED is serious and life-threatening disorder secondary to the complications of hyperthermia and respiratory tract infections in the first years of life, followed by significant and life-long health and quality of life issues (Pavlis, M. B. et al., Pediatr Dermatol. 2010 May-June; 27(3):260-5). There is no satisfactory treatment that has been approved for patients affected by XLHED.

Correction, alteration and/or mitigation of the phenotypic presentations associated with XLHED in animal models has been accomplished by the administration of a recombinant form of the ligand for the EDA receptor. Such recombinant compositions previously identified include those described in detail in U.S. patent application Ser. No. 12/756,268 filed Apr. 8, 2010 which is a continuation of U.S. patent application Ser. No. 10/503,999 filed Oct. 25, 2004, now granted U.S. Pat. No. 7,736,657, which is a 35 U.S.C. Section 371 National Phase Entry Application of International Application No. PCT/EP2002/009354 filed Aug. 21, 2002, which designates the U.S., and which claims the benefit of priority of German Application No. 10205368.5 filed Feb. 10, 2002 and German Application No. 10205583.1 filed Feb. 11, 2002, the contents of which are incorporated herein by reference in their entireties.

The present invention provides recombinant amino-acid based compounds and compositions distinct from those in the art and which comprise EDI200 monomers, multimers, variants, fragments and/or combinations of the foregoing. Further provided are methods of treating persons having or suspected of having a disease, condition or disorder of the ectoderm with a pharmaceutical composition comprising such EDI200 monomers, multimers, variants or fragments.

### SUMMARY OF THE INVENTION

According to the present invention, methods and compositions are provided for the administration of EDA agonists, in particular EDI200, to treat and/or alter one or more phenotypic presentations of ectodermal dysplasia in humans and specifically in the treatment and/or amelioration of conditions associated with XLHED.

In some embodiments, the present invention comprises a pharmaceutical composition comprising EDI200 and a pharmaceutically acceptable excipient. EDI200 may comprises at least one protein monomer, two protein monomers, three protein monomers, four protein monomers, five protein monomers or six protein monomers where the monomer is described by SEQ ID NO. 1.

The EDI200 monomers may be glycosylated, sialylated or otherwise post translationally modified. Glycosylation may occur on any amino acid. In some embodiments glycosylation occurs on one or more asparagine residues. In some embodiments, glycosylation occurs on Asn76 and/or Asn302.

According to the present invention, methods are provided for the treatment of a disease or condition with a pharmaceutical composition comprising one or more EDI200 polypeptides. Such disease or condition may be an ectodermal dysplasia. In some embodiments the ectodermal dysplasia is caused by a deficiency in EDA-A1. In other embodiments, the ectodermal dysplasia is caused by a missense, nonsense or other alteration in the EDA Receptor gene and/or protein. In some embodiments, certain phenotypic presentations or manifestations of an ectodermal dysplasia may be altered by the administration of an EDI200 pharmaceutical composition. These include, but are not limited to, missing teeth, abnormally shaped teeth, abnormal morphology or lack of sweat glands, lack of Meibomian glands, lack of glands of the upper respiratory tract, lack of sebaceous glands, lack of salivary glands, lack or abnormal morphology of various types of hair, and alopecia.

In some embodiments, the ectodermal dysplasia is X-linked hypohidrotic ectodermal dysplasia (XLHED).

Dosing of EDI200 pharmaceutical compositions may be in unit dosage form with a pharmaceutically acceptable excipient or delivery agent.

In some embodiments the excipient is a diluent comprising sodium phosphate and sodium chloride. It may further comprise one or more surfactants and/or detergents.

In some embodiments the pharmaceutical composition comprises about 0.5% EDI200, about 20 mM sodium phosphate, about 300 mM sodium chloride and about 0.02% polysorbate 20 by volume.

In some embodiments, the unit dose is from about 1 mg/kg to about 200 mg/kg. In some embodiments, the unit dose is from about 1 mg/kg to about 100 mg/kg.

In some embodiments are provided methods for correcting, altering or mitigating one or more phenotypic presentations of ectodermal dysplasia in a human diagnosed with or suspected of having ectodermal dysplasia comprising, administering to said human a pharmaceutical composition comprising EDI200. The EDI200 pharmaceutical composition may be administered by intravenous injection using continuous infusion wherein the infusion rate is selected from the group consisting of from about 0.1 ml/kg/hour to about 1 ml/kg/hour, from about 0.5 ml/kg/hour to about 5 ml/kg/hour, from about 1.5 ml/kg/hour to about 10 ml/kg/hour or from about 3 ml/kg/hour to about 20 ml/kg/hour and the continuous infusion may occur over a period of time selected from the group consisting of from about 1 min to about 1 hour, from about 5 min to about 2 hours, from about 10 min to about 3 hours, from about 30 min to about 4 hours, from about 45 min to about 5 hours and at least 5 hours.

In some embodiments administration to a human is via in utero administration to the human's mother. In some embodiments, administration is directly into the amniotic fluid. In some embodiments, administration is selected from the group consisting of the cavity of the amnion, the cavity of the uterus, the μmbilical cord, the placenta, placental vilii, any structure, lumen cavity or vessel associated with gestation. In this embodiment, administration may occur anytime during the pregnancy. Administration may also occur immediately after birth of the human and/or through childhood and/or in adulthood.

In some embodiments, administration to the individual is through the milk of the affected subject's lactating mother. In this embodiment, administration is to the mother either during pregnancy or after pregnancy and for aduration sufficient to deliver the EDI200 drug substance to the affected offspring for treatment of an ectodermal dysplasia, specifically XLHED. The duration of administration to the mother may be over hours, days, weeks or months.

In some embodiments, the mother is tested via methods in the art, such as amniocentesis, prior to administration. In some embodiment, family members of the mother or the affected individual are tested for markers, genotypes, patterns or evaluated for phenotypic presentations prior to administration of the compounds or compositions of the invention.

### DETAILED DESCRIPTION

Unless otherwise defined, all technical and scientific terms used herein have the same meaning as commonly understood by one of ordinary skill in the art to which this invention belongs. Although methods and materials similar or equivalent to those described herein can be used in the practice or testing of methods featured in the invention, suitable methods and materials are described below in the detailed description, examples and claims.

The present invention provides pharmaceutical compounds, compositions, combinations and formulations, methods, assays and kits for treating, correcting, altering, mitigating and/or modifying the etiology, clinical presentation or one or more symptoms of ectodermal dysplasia, specifically in an individual diagnosed with or suffering from XLHED.

In one embodiment of the invention is a pharmaceutical composition comprising EDI200. EDI200 is a fully humanized Fc fusion protein consisting of the Fc region of human IgG1 and the receptor binding domain (Tumor Necrosis Factor (TNF) domain) of EDA-A1.

In some embodiments, the biologically active protein composition is glycosylated and exists primarily as a hexamer comprised of six identical monomeric species with an approximate molecular weight of 290 kDa. The sequence of the monomeric species of EDI200 is provided herein as SEQ ID NO: 1.

### Compounds and Compositions of the Invention

The present invention provides recombinant amino-acid based (e.g., polypeptide) compounds and compositions which comprise EDI200 monomers, multimers, variants, fragments and/or combinations of the foregoing.

According to the present invention, the term “EDI200” refers to a fully humanized fusion protein between the C-terminus of a human immunoglobulin G constant region (IgG Fc) and the receptor-binding domain (Tumor Necrosis Factor (TNF) domain of human EDA-A1. EDI200 exists primarily as a glycosylated hexamer comprised of six identical monomeric polypetides. The monomeric polypeptide is represented by SEQ ID NO: 1.

In some embodiments, EDI200 exists exclusively as a hexamer. In some embodiments EDI200 exists in at least 80%, at least 90%, at least 95%, at least 98% or greater than 99% hexameric form and still remains active.

EDI200 compounds and compositions of the present invention may exist as a single polypeptide monomer, a plurality of polypeptides or fragments of polypeptides, which independently may be encoded by one or more nucleic acids, a plurality of nucleic acids, fragments of nucleic acids or variants of any of the aforementioned.

As used herein, “polypeptide” means a polymer of amino acid residues (natural or unnatural) linked together most often by peptide bonds. The term, as used herein, refers to proteins, polypeptides, and peptides of any size, structure, or function. In some instances the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and preferably, they will be at least about 80%, more preferably at least about 90% identical (homologous) to a native or reference sequence.

In some embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

“Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

As used herein as it applies to polypeptide sequences, the term “homologs” refers to polypeptide sequences having substantial identity between two or more species.

“Analogs” is meant to include polypeptide variants which differ by one or more amino acid alterations, e.g., substitutions, additions or deletions of amino acid residues that still maintain one or more of the properties of the parent or starting polypeptide.

The present invention contemplates several types of compositions which are polypeptide based including variants and derivatives. These include substitutional, insertional, deletion and covalent variants and derivatives. The term “derivative” is used synonymously with the term “variant” but generally refers to a molecule that has been modified and/or changed in any way relative to a reference molecule or starting molecule.

As such, polypeptides containing substitutions, insertions and/or additions, deletions and covalent modifications with respect to reference sequences, in particular the polypeptide sequences disclosed herein, are included within the scope of this invention. For example, sequence tags or amino acids, such as one or more lysines, can be added to the peptide sequences of the invention (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble, or linked to a solid support.

“Substitutional variants” when referring to polypeptides are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine and leucine for another non-polar residue. Likewise, examples of conservative substitutions include the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

“Insertional variants” when referring to polypeptides are those with one or more amino acids inserted immediately adjacent to an amino acid at a particular position in a native or starting sequence. “Immediately adjacent” to an amino acid means connected to either the alpha-carboxy or alpha-amino functional group of the amino acid.

“Deletional variants” when referring to polypeptides are those with one or more amino acids in the native or starting amino acid sequence removed. Ordinarily, deletional variants will have one or more amino acids deleted in a particular region of the molecule.

“Covalent derivatives” when referring to polypeptides include modifications of a native or starting protein with an organic proteinaceous or non-proteinaceous derivatizing agent, and/or post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant protein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the polypeptides produced in accordance with the present invention.

Other post-translational modifications include hydroxylation of proline and lysine, phosphorylation of hydroxyl groups of seryl or threonyl residues, methylation of the alpha-amino groups of lysine, arginine, and histidine side chains (Creighton, T. E., Proteins: Structure and Molecular Properties, W.H. Freeman & Co., San Francisco, 1983 , pp. 79-86).

“Features” when referring to polypeptides are defined as distinct amino acid sequence-based components of a molecule. Features of the polypeptides of the present invention include surface manifestations, local conformational shape, folds, loops, half-loops, domains, half-domains, sites, termini or any combination thereof.

As used herein when referring to polypeptides the term “surface manifestation” refers to a polypeptide based component of a protein appearing on an outermost surface.

As used herein when referring to polypeptides the term “local conformational shape” means a polypeptide based structural manifestation of a protein which is located within a definable space of the protein.

As used herein when referring to polypeptides the term “fold” refers to the resultant conformation of an amino acid sequence upon energy minimization. A fold may occur at the secondary or tertiary level of the folding process. Examples of secondary level folds include beta sheets and alpha helices. Examples of tertiary folds include domains and regions formed due to aggregation or separation of energetic forces. Regions formed in this way include hydrophobic and hydrophilic pockets, and the like.

As used herein the term “turn” as it relates to protein conformation means a bend which alters the direction of the backbone of a peptide or polypeptide and may involve one, two, three or more amino acid residues.

As used herein when referring to polypeptides the term “loop” refers to a structural feature of a polypeptide which may serve to reverse the direction of the backbone of a peptide or polypeptide. Where the loop is found in a polypeptide and only alters the direction of the backbone, it may comprise four or more amino acid residues. Oliva et al. have identified at least 5 classes of protein loops (Oliva, B. et al., J Mol Biol. 1997 Mar. 7; 266(4):814-30). Loops may be open or closed. Closed loops or “cyclic” loops may comprise 2, 3, 4, 5, 6, 7, 8, 9, 10 or more amino acids between the bridging moieties. Such bridging moieties may comprise a cysteine-cysteine bridge (Cys-Cys) typical in polypeptides having disulfide bridges or alternatively bridging moieties may be non-protein based such as the dibromozylyl agents used herein.

As used herein when referring to polypeptides the term “half-loop” refers to a portion of an identified loop having at least half the number of amino acid resides as the loop from which it is derived. It is understood that loops may not always contain an even number of amino acid residues. Therefore, in those cases where a loop contains or is identified to comprise an odd number of amino acids, a half-loop of the odd-numbered loop will comprise the whole number portion or next whole number portion of the loop (number of amino acids of the loop/2+/−0.5 amino acids). For example, a loop identified as a 7 amino acid loop could produce half-loops of 3 amino acids or 4 amino acids (7/2=3.5+/−0.5 being 3 or 4).

As used herein when referring to polypeptides the term “domain” refers to a motif of a polypeptide having one or more identifiable structural or functional characteristics or properties (e.g., binding capacity, serving as a site for protein-protein interactions).

As used herein when referring to polypeptides the term “half-domain” means a portion of an identified domain having at least half the number of amino acid resides as the domain from which it is derived. It is understood that domains may not always contain an even number of amino acid residues. Therefore, in those cases where a domain contains or is identified to comprise an odd number of amino acids, a half-domain of the odd-numbered domain will comprise the whole number portion or next whole number portion of the domain (number of amino acids of the domain/2+/−0.5 amino acids). For example, a domain identified as a 7 amino acid domain could produce half-domains of 3 amino acids or 4 amino acids (7/2=3.5+/−0.5 being 3 or 4). It is also understood that sub-domains may be identified within domains or half-domains, these subdomains possessing less than all of the structural or functional properties identified in the domains or half domains from which they were derived. It is also understood that the amino acids that comprise any of the domain types herein need not be contiguous along the backbone of the polypeptide (i.e., nonadjacent amino acids may fold structurally to produce a domain, half-domain or subdomain).

As used herein when referring to polypeptides the terms “site” as it pertains to amino acid based embodiments is used synonymously with “amino acid residue” and “amino acid side chain.” A site represents a position within a peptide or polypeptide that may be modified, manipulated, altered, derivatized or varied within the polypeptide based molecules of the present invention.

As used herein the terms “termini” or “terminus” when referring to polypeptides refers to an extremity of a peptide or polypeptide. Such extremity is not limited only to the first or final site of the peptide or polypeptide but may include additional amino acids in the terminal regions. The polypeptide based molecules of the present invention may be characterized as having both an N-terminus (terminated by an amino acid with a free amino group (NH2)) and a C-terminus (terminated by an amino acid with a free carboxyl group (COOH)). Proteins of the invention are in some cases made up of multiple polypeptide chains brought together by disulfide bonds or by non-covalent forces (multimers, oligomers). These sorts of proteins will have multiple N- and C-termini. Alternatively, the termini of the polypeptides may be modified such that they begin or end, as the case may be, with a non-polypeptide based moiety such as an organic conjugate.

Once any of the features have been identified or defined as a desired component of a polypeptide, any of several manipulations and/or modifications of these features may be performed by moving, swapping, inverting, deleting, randomizing or duplicating. Furthermore, it is understood that manipulation of features may result in the same outcome as a modification to the molecules of the invention. For example, a manipulation which involved deleting a domain would result in the alteration of the length of a molecule just as modification of a nucleic acid to encode less than a full length molecule would.

Modifications and manipulations can be accomplished by methods known in the art such as, but not limited to, site directed mutagenesis. The resulting modified molecules may then be tested for activity using in vitro or in vivo assays such as those described herein or any other suitable screening assay known in the art.

According to the present invention, the polypeptides may comprise a consensus sequence which is discovered through rounds of experimentation. As used herein a “consensus” sequence is a single sequence which represents a collective population of sequences allowing for variability at one or more sites.

As recognized by those skilled in the art, protein fragments, functional protein domains, and homologous proteins are also considered to be within the scope of polypeptides of this invention. For example, provided herein is any protein fragment (meaning a polypeptide sequence at least one amino acid residue shorter than a reference polypeptide sequence but otherwise identical) of a reference protein 10, 20, 30, 40, 50, 60, 70, 80, 90, 100 or greater than 100 amino acids in length. In another example, any protein that includes a stretch of about 20, about 30, about 40, about 50, or about 100 amino acids which are about 40%, about 50%, about 60%, about 70%, about 80%, about 90%, about 95%, or about 100% identical to any of the sequences described herein can be utilized in accordance with the invention. In certain embodiments, a polypeptide to be utilized in accordance with the invention includes 2, 3, 4, 5, 6, 7, 8, 9, 10, or more mutations as shown in any of the sequences provided or referenced herein.

The term “identity” as known in the art, refers to a relationship between the sequences of two or more peptides, as determined by comparing the sequences. In the art, identity also means the degree of sequence relatedness between peptides, as determined by the number of matches between strings of two or more amino acid residues. Identity measures the percent of identical matches between the smaller of two or more sequences with gap alignments (if any) addressed by a particular mathematical model or computer program (i.e., “algorithms”). Identity of related peptides can be readily calculated by known methods. Such methods include, but are not limited to, those described previously by others (Lesk, A. M., ed., Computational Molecular Biology, Oxford University Press, New York, 1988; Smith, D. W., ed., Biocomputing: Informatics and Genome Projects, Academic Press, New York, 1993; Griffin, A. M. et al., ed., Computer Analysis of Sequence Data, Part 1, Humana Press, New Jersey, 1994; von Heinje, G., Sequence Analysis in Molecular Biology, Academic Press, 1987; Gribskov, M. et al., ed., Sequence Analysis Primer, M. Stockton Press, New York, 1991; and Carillo et al., Applied Math, SIAM J, 1988, 48, 1073).

In some embodiments, the polypeptide variant may have the same or a similar activity as the reference polypeptide. Alternatively, the variant may have an altered activity (e.g., increased or decreased) relative to a reference polypeptide. Generally, variants of a particular polynucleotide or polypeptide of the invention will have at least about 40%, 45%, 50%, 55%, 60%, 65%, 70%, 75%, 80%, 85%, 90%, 91%, 92%, 93%, 94%, 95%, 96%, 97%, 98%, 99% but less than 100% sequence identity to that particular reference polynucleotide or polypeptide as determined by sequence alignment programs and parameters described herein and known to those skilled in the art. Such tools for alignment include those of the BLAST suite (Altschul, S. F. et al., Gapped BLAST and PSI-BLAST: a new generation of protein database search programs, Nucleic Acids Res. 1997, 25:3389-3402) Other tools are described herein, specifically in the definition of “Identity.”

Default parameters in the BLAST algorithm include, for example, an expect threshold of 10, Word size of 28, Match/Mismatch Scores 1, −2, Gap costs Linear. Any filter can be applied as well as a selection for species specific repeats, e.g., Homo sapiens.

### Isotopic Variations

Compounds and compositions, including pharmaceutical compositions, of the present invention may contain one or more atoms that are isotopes. As used herein, the term “isotope” refers to a chemical element that has one or more additional neutrons. In one embodiment, compounds and pharmaceutical compositions of the present invention may be deuterated. As used herein, the term “deuterated” refers to a substance that has had one or more hydrogen atoms replaced by deuterium or tritium isotopes. Deuterium and tritium are isotopes of hydrogen. The nucleus of hydrogen contains one proton while deuterium nuclei contain both a proton and a neutron. Compounds and pharmaceutical compositions of the present invention may be deuterated in order to change a physical property, such as stability, or to allow them to be used in diagnostic and experimental applications.

### Pharmaceutical Composition and Formulations

The present invention provides EDI200 and variations thereof as well as compositions and complexes comprising one or more pharmaceutically acceptable excipients. Pharmaceutical compositions may optionally comprise one or more additional active substances, e.g. therapeutically and/or prophylactically active substances. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, compositions are administered to humans, human patients or subjects. For the purposes of the present disclosure, the phrase “active ingredient” generally refers to EDI200 or variations thereof to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals. Subjects to which administration of the pharmaceutical compositions is contemplated include, but are not limited to, humans and/or other primates and mammals, including commercially relevant mammals.

### Formulations

Compounds and pharmaceutical compositions of the invention can be formulated using one or more excipients to: (1) increase stability; (2) permit the sustained or delayed release; (3) alter the biodistribution (e.g., target active ingredients to specific tissues or cell types); and (4) alter the release profile of the drug in vivo. Formulations of compounds and pharmaceutical compositions described herein may be prepared by any method known or hereafter developed in the art of pharmacology. In general, such preparatory methods include a step associating active ingredients with excipient and/or one or more accessory ingredients.

### Excipients

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants, lipidoids, liposomes, lipid nanoparticles, polymers, lipoplexes, core-shell nanoparticles, peptides, proteins, and combinations thereof as suited to the particular dosage form desired. Remington's The Science and Practice of Pharmacy, 21s^{t} Edition, A. R. Gennaro (Lippincott, Williams & Wilkins, Baltimore, Md., 2006; incorporated herein by reference) discloses various excipients used in formulating pharmaceutical compositions and known techniques for the preparation thereof.

Except insofar as any conventional excipient is incompatible with a substance or its derivatives, such as by producing any undesirable biological effect or otherwise interacting in a deleterious manner with any other component(s) of the pharmaceutical composition, its use is contemplated to be within the scope of this invention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical compositions.

In some embodiments, diluents may comprise calcium carbonate, sodium carbonate, calcium phosphate, dicalcium phosphate, calcium sulfate, calcium hydrogen phosphate, sodium phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM®), sodium lauryl sulfate, quaternary ammonium compounds, etc., and/or combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and VEEGUM® [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [TWEEN®20], polyoxyethylene sorbitan [TWEENn®60], polyoxyethylene sorbitan monooleate [TWEEN®80], sorbitan monopalmitate [SPAN®40], sorbitan monostearate [Span®60], sorbitan tristearate [Span 65], glyceryl monooleate, sorbitan monooleate [SPAN®80]), polyoxyethylene esters (e.g. polyoxyethylene monostearate [MYRJ®45], polyoxyethylene hydrogenated castor oil, polyethoxylated castor oil, polyoxymethylene stearate, and SOLUTOL®), sucrose fatty acid esters, polyethylene glycol fatty acid esters (e.g. CREMOPHOR®), polyoxyethylene ethers, (e.g. polyoxyethylene lauryl ether [BRIJ®30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, PLUORINC®F 68, POLOXAMER®188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan); alginates; polyethylene oxide; polyethylene glycol; inorganic calcium salts; silicic acid; polymethacrylates; waxes; water; alcohol; etc.; and combinations thereof.

Exemplary preservatives may include, but are not limited to, antioxidants, chelating agents, antimicrobial preservatives, antifungal preservatives, alcohol preservatives, acidic preservatives, and/or other preservatives.

Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and/or sodium sulfite.

Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and/or trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and/or thimerosal.

Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and/or sorbic acid.

Exemplary alcohol preservatives include, but are not limited to, ethanol, polyethylene glycol, phenol, phenolic compounds, bisphenol, chlorobutanol, hydroxybenzoate, and/or phenylethyl alcohol.

Exemplary acidic preservatives include, but are not limited to, vitamin A, vitamin C, vitamin E, beta-carotene, citric acid, acetic acid, dehydroacetic acid, ascorbic acid, sorbic acid, and/or phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, GLYDANT PLUS®, PHENONIP®, methylparaben, GERMALL®115, GERMABEN®II, NEOLONE™, KATHON™, and/or EUXYL®.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and/or combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and/or combinations thereof.

Excipients such as cocoa butter and suppository waxes, coloring agents, coating agents, sweetening, flavoring, and/or perfuming agents can be present in the composition, according to the judgment of the formulator.

Compounds and pharmaceutical compositions in accordance with the present disclosure may be prepared, packaged, and/or sold in bulk, as a single unit dose, and/or as a plurality of single unit doses. As used herein, a “unit dose” refers to a discrete amount of compounds or pharmaceutical compositions comprising a predetermined amount of active ingredient. The amount of active ingredient may generally be equal to active ingredient dosage administered to subjects and/or convenient fractions of such dosages including, but not limited to, one-half or one-third of such dosages.

Relative amounts of active ingredient, pharmaceutically acceptable excipients, and/or any additional ingredients in pharmaceutical compositions in accordance with the present disclosure may vary, depending upon the identity, size, and/or condition of subjects being treated and further depending upon administration route. For example, compositions may comprise from about 0.001% to about 100%, from about 0.01% to about 3.0%, from about 0.02% to about 4%, from about 0.05% to about 10%, from about 0.10% to about 20%, from about 0.15% to about 75%, from about 0.30% to about 60%, from about 0.50% to about 50%, from about 1.0% to about 30%, from about 5% to about 80%, or at least 80% (w/w) active ingredient. In some embodiments, the active ingredient is EDI200 (e.g., including monomer or multimeric forms) or a fragment or variant thereof.

### Polymers and Matrices

Compounds and pharmaceutical compositions of the invention can be formulated with or within natural and/or synthetic polymers. Additionally polymers may be biodegradable or non-biodegradable depending on their composition. Non-limiting examples of biodegradable polymers which may be used for delivery include, but are not limited to, protein-based polymers (including, but not limited to collagen, albumin and gelatin), polysaccharides (including, but not limited to agarose, alginate, carrageenan, hyaluronic acid, dextran, chitosan and cyclodextrins), polyesters (including, but not limited to poly(lactic acid), poly(glycolic acid), polyesters derived from lactic and glycolic acids (PLGA), poly(hydroxyl butyrate), poly(epsilon-caprolactone), poly(alpha-malic acid) and poly(dioxanones)), polyanhydrides (including, but not limited to poly(sebacic acid), poly(adipic acid), poly(terphthalic acid) and various copolymers), polyamides (including, but not limited to poly(imino carbonates) and polyamino acids), phosphorous-based polymers (including, but not limited to polyphosphates, polyphosphonates and polyphosphazenes), poly(cyano acrylates), polyurethanes, polyortho esters, polydihydropyrans and polyacetals. Non-limiting examples of non-biodegradable polymers which may be used for delivery include, but are not limited to, cellulose derivatives (including, but not limited to, carboxymethyl cellulose, ethyl cellulose, cellulose acetate, cellulose acetate propionate and hydroxyporpyl methylcellulose), silicones (including, but not limited to, polydimethylsiloxane, colloidal silica, polymethacrylates, poly(methyl methacrylate) and poly hydro(ethylmethacrylate)), polyvinyl pyrrolidone, ethyl vinyl acetate, poloxamers and poloxamines.

Polymer formulations may permit the sustained or delayed release of compounds of the invention (e.g., following intramuscular or subcutaneous injection). The altered release profile may result in, for example, receptor activation over an extended period of time. The polymer formulation may also be used to increase the stability of active ingredients. In one embodiment, the pharmaceutical compositions may be sustained release formulations. In a further embodiment, the sustained release formulations may be for subcutaneous delivery. Sustained release formulations may include, but are not limited to, PLGA microspheres, ethylene vinyl acetate (EVAc), poloxamer, GELSITE® (Nanotherapeutics, Inc. Alachua, Fla.), HYLENEX® (Halozyme Therapeutics, San Diego Calif.), surgical sealants such as fibrinogen polymers (Ethicon Inc. Cornelia, Ga.). TISSELL® (Baxter International, Inc Deerfield, Ill.), PEG-based sealants, and COSEAL® (Baxter International, Inc Deerfield, Ill.).

As a non-limiting example, compounds and pharmaceutical compositions of the invention may be formulated in PLGA microspheres by preparing the PLGA microspheres with tunable release rates (e.g., days and weeks) and encapsulating compounds and pharmaceutical compositions in the PLGA microspheres while maintaining their integrity during the encapsulation process. EVAc are non-biodegradeable, biocompatible polymers which are used extensively in pre-clinical sustained release implant applications (e.g., extended release products Ocusert a pilocarpine ophthalmic insert for glaucoma or progestasert a sustained release progesterone intrauterine device; transdermal delivery systems Testoderm, Duragesic and Selegiline; catheters). Poloxamer F-407 NF is a hydrophilic, non-ionic surfactant triblock copolymer of polyoxyethylene-polyoxypropylene-polyoxyethylene having a low viscosity at temperatures less than 5° C. and forms a solid gel at temperatures greater than 15° C. Polyethylene glycol (PEG)-based surgical sealants comprise two synthetic PEG components mixed in a delivery device which can be prepared in one minute, seals in 3 minutes and is reabsorbed within 30 days. GELSITE® and natural polymers are capable of in-situ gelation at the site of administration. They have been shown to interact with protein and peptide therapeutic candidates through ionic interaction to provide a stabilizing effect.

Polymer formulations may also be selectively targeted through expression of different ligands as exemplified by, but not limited by, folate, transferrin, and N-acetylgalactosamine (GalNAc) (Benoit et al., Biomacromolecules. 2011 12:2708-2714; Rozema et al., Proc Natl Acad Sci USA. 2007 104:12982-12887; Davis, Mol Pharm. 2009 6:659-668; Davis, Nature 2010 464:1067-1070; herein incorporated by reference in its entirety).

### Cells

Polynucleotides encoding compounds of the invention may be transfected ex vivo into cells, which are subsequently transplanted into a subject. In some embodiments, red blood cells, viral particles and/or electroporated cells are used to deliver payloads according to methods that have been documented (Godfrin, Y. et al., Expert Opin Biol Ther. 2012 12:127-133; Fang, R. H. et al., Expert Opin Biol Ther. 2012 April; 12(4):385-9; Hu, C. M. et al., Proc Natl Acad Sci USA. 2011 Jul. 5; 108(27):10980-5; all of which are herein incorporated by reference in their entirety). Cell-based formulations of compounds of the invention may be used to alter the biodistribution of the compound (e.g., by targeting the cell carrier to specific tissues or cell types).

A variety of methods are known in the art and are suitable for introducing polynucleotides encoding compounds of the invention into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion.

The technique of sonoporation, or cellular sonication, is the use of sound (e.g., ultrasonic frequencies) for modifying the permeability of the cell plasma membrane. Sonoporation methods are known to those in the art and are used to deliver nucleic acids in vivo (Yoon and Park, Expert Opin Drug Deliv. 2010 7:321-330; Postema and Gilja, Curr Pharm Biotechnol. 2007 8:355-361; Newman and Bettinger, Gene Ther. 2007 14:465-475; all herein incorporated by reference in their entirety). Sonoporation methods are known in the art and are also taught for example as it relates to bacteria in US Patent Publication 20100196983 and as it relates to other cell types in, for example, US Patent Publication 20100009424, each of which are incorporated herein by reference in their entirety.

Electroporation techniques are also well known in the art and are used to deliver nucleic acids in vivo and clinically (Andre et al., Curr Gene Ther. 2010 10:267-280; Chiarella et al., Curr Gene Ther. 2010 10:281-286; Hojman, Curr Gene Ther. 2010 10:128-138; all herein incorporated by reference in their entirety).

### Hyaluronidase

The intramuscular or subcutaneous localized injection of compounds of the invention can include hyaluronidase, which catalyzes the hydrolysis of hyaluronan. By catalyzing the hydrolysis of hyaluronan, a constituent of the interstitial barrier, hyaluronidase lowers the viscosity of hyaluronan, thereby increasing tissue permeability (Frost, Expert Opin. Drug Deliv. (2007) 4:427-440; herein incorporated by reference in its entirety). It is useful to speed the dispersion and systemic distribution of the injected compounds. Alternatively, the hyaluronidase can be used to increase the number of cells exposed to compounds of the invention administered intramuscularly or subcutaneously.

### Formulated Delivery

Compounds and pharmaceutical compositions of the present invention may be formulated, using the methods described herein. The formulations may contain compounds which may be modified and/or unmodified. The formulations may further include, but are not limited to pharmaceutically acceptable carriers, delivery agents, bioerodible and/or biocompatible polymers, solvents, and sustained-release delivery depots. The formulated compounds may be delivered using routes of administration known in the art and described herein.

Compounds and pharmaceutical compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like.

In some embodiments, pharmaceutical compositions and formulations include EDI200 compounds. In some embodiments, treatment regimens comprise combinations of compounds or combinations of treatment regimens, each of which comprise administration of a pharmaceutical composition comprising EDI200. Compounds and pharmaceutical compositions of the present invention may be administered in a number of ways depending upon whether local or systemic treatment is desired and upon the area to be treated. Compounds and pharmaceutical compositions of the present invention may be administered using a combination of methodologies.

As a non-limiting example, pharmaceutical compositions may be administered by intravenous injection or infusion and/or intraperitoneal injection. Administration may be topical (e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. EDI200 may be delivered in a manner to target a particular tissue.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful. Suitable topical formulations include those in which EDI200 is in an admixture with a topical delivery agent such as lipids, liposomes, fatty acids, fatty acid esters, steroids, chelating agents and surfactants.

In one embodiment of the invention, EDI200 is formulated for intraveneous infusion with a pH 7.2 solution comprising 20 mM sodium phosphate, 300 mM NaCl and about 0.02% polysorbate 20 (e.g., commercial brand; TWEEN®20)).

### Administration and Dosing

In some embodiments, organisms to be treated may be mammals, including, but not limited to humans. In such embodiments, compounds and pharmaceutical compositions may be administered by any route which results in therapeutically effective outcomes including, but not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops.

In some embodiments, compositions may be administered in a way which allows them cross the blood-brain barrier, vascular barrier, or other epithelial barrier. In certain embodiments, the compositions are administered by intravenous infusion or injection. Non-limiting routes of administration for compounds and pharmaceutical compositions of the present invention are described below.

### Parenteral and Injectible Administration

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such as CREMOPHOR®, alcohols, oils, modified oils, glycols, polysorbates (including, but not limited to polysorbate-20), cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In order to prolong the effect of an active ingredient, it is often desirable to slow the absorption of the active ingredient from subcutaneous or intramuscular injection. This may be accomplished by the use of a liquid suspension of crystalline or amorphous material with poor water solubility. The rate of absorption of the active ingredient then depends upon its rate of dissolution which, in turn, may depend upon crystal size and crystalline form. Alternatively, delayed absorption of a parenterally administered compound or pharmaceutical composition is accomplished by dissolving or suspending the active ingredient in an oil vehicle. Injectable depot forms are made by forming microencapsule matrices of the compounds or pharmaceutical compositions in biodegradable polymers such as polylactide-polyglycolide. Depending upon the ratio of active ingredient to polymer and the nature of the particular polymer employed, the rate of release can be controlled. Examples of other biodegradable polymers include poly(orthoesters) and poly(anhydrides). Depot injectable formulations are prepared by entrapping the compounds or pharmaceutical compositions in liposomes or microemulsions which are compatible with body tissues.

In one embodiment, EDI200 is formulated for injection in a pH 7.2 buffer comprising 20 mM sodium phosphate, 300 mM NaCl and 0.02% Polysorbate 20 (or about 0.2% TWEEN®20). In another embodiment, compounds of the invention are administered by injection via percutaneous peripheral vein catheter. In another embodiment, compounds and pharmaceutical compositions of the invention are administered by infusion at a rate from about 0.1 ml/kg/hour to about 1 ml/kg/hour, from about 0.5 ml/kg/hour to about 5 ml/kg/hour, from about 1.5 ml/kg/hour to about 10 ml/kg/hour or from about 3 ml/kg/hour to about 20 ml/kg/hour. In a further embodiment, infusion time is from about 1 min to about 1 hour, from about 5 min to about 2 hours, from about 10 min to about 3 hours, from about 30 min to about 4 hours, from about 45 min to about 5 hours or at least 5 hours.

In some embodiments, compounds and pharmaceutical compositions of the invention are administered by infusion at standard room temperature. “Standard room temperature” as used herein means a temperature between 15°-25° Celsius, including but not limited to 15.0°, 15.5°, 16.0°, 16.5°, 17.0°, 17.5°, 18.0°, 18.5°, 19.0°, 19.5°, 20.0°, 20.5°, 21.0°, 21.5°, 22.0°, 22.5°, 23.0°, 23.5°, 24.0°, 24.5°, 25.0° C. is considered room temperature. In some embodiments, the pharmaceutical composition may be brought to standard room temperature. In some embodiments, the administration of the pharmaceutical composition may occur at standard room temperature, irrespective of the temperature of the pharmaceutical composition itself. In some embodiments, the administration device, for example the infusion system or apparatus may be held or maintained at or around room temperature, either with or without regard to the ambient temperature or the temperature of the pharmaceutical composition.

### Rectal and Vaginal Administration

Compositions for rectal or vaginal administration are typically suppositories which can be prepared by mixing compositions with suitable non-irritating excipients such as cocoa butter, polyethylene glycol or a suppository wax which are solid at ambient temperature but liquid at body temperature and therefore melt in the rectum or vaginal cavity and release the active ingredient.

### Oral Administration

Solid dosage forms for oral administration include capsules, tablets, pills, powders, and granules. In such solid dosage forms, an active ingredient is mixed with at least one inert, pharmaceutically acceptable excipient such as sodium citrate or dicalcium phosphate and/or fillers or extenders (e.g. starches, lactose, sucrose, glucose, mannitol, and silicic acid), binders (e.g. carboxymethylcellulose, alginates, gelatin, polyvinylpyrrolidinone, sucrose, and acacia), humectants (e.g. glycerol), disintegrating agents (e.g. agar, calcium carbonate, potato or tapioca starch, alginic acid, certain silicates, and sodium carbonate), solution retarding agents (e.g. paraffin), absorption accelerators (e.g. quaternary ammonium compounds), wetting agents (e.g. cetyl alcohol and glycerol monostearate), absorbents (e.g. kaolin and bentonite clay), and lubricants (e.g. talc, calcium stearate, magnesium stearate, solid polyethylene glycols, sodium lauryl sulfate), and mixtures thereof. In the case of capsules, tablets and pills, the dosage form may comprise buffering agents.

### Topical or Transdermal Administration

As described herein, compounds and pharmaceutical compositions of the invention may be formulated for administration topically. The skin may be an ideal target site for delivery as it is readily accessible.

The site of the delivered compositions will depend on the route of delivery. Three routes are commonly considered for delivery to the skin: (i) topical application, (ii) intradermal injection and (iii) systemic delivery. Compounds of the invention can be delivered to the skin by several different approaches known in the art. In one embodiment, the invention provides for a variety of dressings or bandages (e.g., adhesive bandages) for conveniently and/or effectively carrying out methods of the present invention. Typically dressing or bandages may comprise sufficient amounts of pharmaceutical compositions and/or compounds described herein to allow a user to perform multiple treatments of subjects.

In some embodiments, before topical and/or transdermal administration at least one area of tissue, such as skin, may be subjected to a device and/or solution which may increase permeability. In one embodiment, the tissue may be subjected to an abrasion device to increase the permeability of the skin (see U.S. Patent Publication No. 20080275468, herein incorporated by reference in its entirety). In another embodiment, the tissue may be subjected to an ultrasound enhancement device. An ultrasound enhancement device may include, but is not limited to, the devices described in U.S. Publication No. 20040236268 and U.S. Pat. Nos. 6,491,657 and 6,234,990; herein incorporated by reference in their entireties. Methods of enhancing the permeability of tissue are described in U.S. Publication Nos. 20040171980 and 20040236268 and U.S. Pat. No. 6,190,315; herein incorporated by reference in their entireties.

In some embodiments, a device may be used to increase permeability of tissue before delivering formulations of the invention. The permeability of skin may be measured by methods known in the art and/or described in U.S. Pat. No. 6,190,315, herein incorporated by reference in its entirety. As a non-limiting example, a modified mRNA formulation may be delivered by the delivery methods described in U.S. Pat. No. 6,190,315, herein incorporated by reference in its entirety.

In some embodiments, tissue may be treated with a eutectic mixture of local anesthetics (EMLA) cream before, during and/or after the tissue may be subjected to a device which may increase permeability. Katz et al. (Anesth Analg (2004); 98:371-76; herein incorporated by reference in its entirety) showed that using the EMLA cream in combination with a low energy, an onset of superficial cutaneous analgesia was seen as fast as 5 minutes after a pretreatment with a low energy ultrasound.

In some embodiments, enhancers may be applied to the tissue before, during, and/or after the tissue has been treated to increase permeability. Enhancers include, but are not limited to, transport enhancers, physical enhancers, and cavitation enhancers. Non-limiting examples of enhancers are described in U.S. Pat. No. 6,190,315, herein incorporated by reference in its entirety.

In some embodiments, a device may be used to increase permeability of tissue before delivering formulations of the invention as described herein, which may further contain a substance that invokes an immune response. In another non-limiting example, a formulation containing a substance to invoke an immune response may be delivered by the methods described in U.S. Publication Nos. 20040171980 and 20040236268; herein incorporated by reference in their entireties.

Dosage forms for topical and/or transdermal administration of a composition may include ointments, pastes, creams, lotions, gels, powders, solutions, sprays, inhalants and/or patches. Generally, an active ingredient is admixed under sterile conditions with a pharmaceutically acceptable excipient and/or any needed preservatives and/or buffers as may be required. Additionally, the present invention contemplates the use of transdermal patches, which often have the added advantage of providing controlled delivery of a compound to the body. Such dosage forms may be prepared, for example, by dissolving and/or dispensing the compound in the proper medium. Alternatively or additionally, rate may be controlled by either providing a rate controlling membrane and/or by dispersing the compound in a polymer matrix and/or gel.

Formulations suitable for topical administration include, but are not limited to, liquid and/or semi liquid preparations such as liniments, lotions, oil in water and/or water in oil emulsions such as creams, ointments and/or pastes, and/or solutions and/or suspensions.

Topically-administrable formulations may, for example, comprise from about 0.1% to about 100% (w/w) active ingredient, although the concentration of active ingredient may be as high as the solubility limit of the active ingredient in the solvent. Formulations for topical administration may further comprise one or more of the additional ingredients described herein.

### Depot Administration

As described herein, in some embodiments, compositions are formulated in depots for extended release. Generally, specific organs or tissues (“target tissues”) are targeted for administration.

In some embodiments, compounds and pharmaceutical compositions of the invention are spatially retained within or proximal to a target tissue. Provided are methods of providing compounds and pharmaceutical compositions to a target tissue of a mammalian subject by contacting the target tissue (which contains one or more target cells) with compounds and pharmaceutical compositions under conditions such that active ingredients are substantially retained in the target tissue, meaning that at least 10, 20, 30, 40, 50, 60, 70, 80, 85, 90, 95, 96, 97, 98, 99, 99.9, 99.99 or greater than 99.99% of active ingredients are retained in the target tissue. For example, intramuscular injection to a mammalian subject is performed using aqueous compositions containing active ingredients of the invention, and retention is determined by measuring the amount of the compound present in the muscle tissue.

In some embodiments, the invention provides for compounds and pharmaceutical compositions of the invention to be delivered in more than one injection or by split dose injections.

In some embodiments, the invention may be retained near target tissue using a small disposable drug reservoir or patch pump. Non-limiting examples of patch pumps include those manufactured and/or sold by BD® (Franklin Lakes, N.J.), Insulet Corporation (Bedford, Mass.), SteadyMed Therapeutics (San Francisco, Calif.), Medtronic (Minneapolis, Minn.), UniLife (York, Pa.), Valeritas (Bridgewater, N.J.), and SpringLeaf Therapeutics (Boston, Mass.).

### Pulmonary Administration

Compounds and pharmaceutical compositions may be prepared, packaged, and/or sold in formulations suitable for pulmonary administration via the buccal cavity. Such formulations may comprise dry particles further comprising active ingredients and which have diameters in the range of from about 0.5 nm to about 7 nm or from about 1 nm to about 6 nm. Such compositions are suitably in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nm and at least 95% of the particles by number have a diameter less than 7 nm. Alternatively, at least 95% of the particles by weight have a diameter greater than 1 nm and at least 90% of the particles by number have a diameter less than 6 nm. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50% to 99.9% (w/w) of the composition, and active ingredient may constitute 0.1% to 20% (w/w) of the composition. A propellant may further comprise additional ingredients such as a liquid non-ionic and/or solid anionic surfactant and/or a solid diluent (which may have a particle size of the same order as particles comprising the active ingredient).

Pharmaceutical compositions formulated for pulmonary delivery may provide an active ingredient in the form of droplets of a solution and/or suspension. Such formulations may be prepared, packaged, and/or sold as aqueous and/or dilute alcoholic solutions and/or suspensions, optionally sterile, comprising active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. Droplets provided by this route of administration may have an average diameter in the range from about 0.1 nm to about 200 nm.

### Intranasal, Nasal and Buccal Administration

Formulations described herein as being useful for pulmonary delivery are useful for intranasal delivery of a pharmaceutical composition. Another formulation suitable for intranasal administration is a coarse powder comprising the active ingredient and having an average particle from about 0.2 μm to 500 μm. Such a formulation is administered in the manner in which snuff is taken, i.e. by rapid inhalation through the nasal passage from a container of the powder held close to the nose.

Formulations suitable for nasal administration may, for example, comprise from about as little as 0.1% (w/w) and as much as 100% (w/w) of active ingredient, and may comprise one or more of the additional ingredients described herein. A pharmaceutical composition may be prepared, packaged, and/or sold in a formulation suitable for buccal administration. Such formulations may, for example, be in the form of tablets and/or lozenges made using conventional methods, and may, for example, contain 0.1% to 20% (w/w) active ingredient, the balance comprising an orally dissolvable and/or degradable composition and, optionally, one or more of the additional ingredients described herein. Alternately, formulations suitable for buccal administration may comprise a powder and/or an aerosolized and/or atomized solution and/or suspension comprising active ingredient. Such powdered, aerosolized, and/or aerosolized formulations, when dispersed, may have an average particle and/or droplet size in the range from about 0.1 nm to about 200 nm, and may further comprise one or more of any additional ingredients described herein.

### Ophthalmic Administration

A pharmaceutical composition may be prepared, packaged, and/or sold in a formulation suitable for ophthalmic administration. Such formulations may, for example, be in the form of eye drops including, for example, a 0.1 to 1.0% (w/w) solution and/or suspension of the active ingredient in an aqueous or oily liquid excipient. Such drops may further comprise buffering agents, salts, and/or one or more other of any additional ingredients described herein. Other ophthalmically-administrable formulations which are useful include those which comprise the active ingredient in microcrystalline form and/or in a liposomal preparation. Ear drops and/or eye drops are contemplated as being within the scope of this invention.

### Administration In Utero

The invention also relates to a method for reversing genetically determined diseases through in utero administration of compounds of the invention. This method can be used in connection with all Placentalia, i.e., vertebrates possessing a placenta, in particular in human and veterinary medicine. Following diagnosis of a genetically determined disease in an embryo, for example by means of chorion biopsy or amniocentesis, or when a genetically determined disease is suspected in an embryo on the basis of the genetic disposition of relations, in particular father and/or mother, the method according to the invention is suitable for already treating the embryo prophylactically and reversing its hereditary phenotype. In one embodiment, the treatment is effected using EDI200 according to the invention, as disclosed above, where appropriate in a corresponding formulation, and is ideally administered to the mother, or the mother animal, at the earliest possible time in the pregnancy. Such administration according to the invention is advantageously parenteral, preferably intravenously or intraarterially.

After EDI200 has been administered, after having been internalized, EDI200 reaches the embryo, typically by way of the placental vessels which connect the embryo to maternal blood circulation.

The dose depends on the genetic disease itself and on the time of the administration (that is on the developmental stage of the embryo), in connection with which the treatment should advantageously start at the earliest possible time in the development of the embryo. EDI200 may be administered at least once, more preferably regularly during the first, second and/or third month of the pregnancy, very particularly preferably, for example, on every second day for a period of at least 14 days in the case of a human embryo, where appropriate, however, at longer intervals as well depending on the dose which is chosen.

In principle, however, the dose of EDI200 according to the invention depends on the method of treatment. In the case of treatment during embryonic development, typical doses of EDI200 are less than one tenth, preferably less than one hundredth, and even more preferably less than one thousandth, of the native concentration of the dose in the neonate. In some embodiments, doses may include, but are not limited to from about 0.0001 mg/kg to about 30 mg/kg, from about 0.00015 mg/kg to about 0.15 mg/kg, from about 0.0003 mg/kg to about 0.3 mg/kg, from about 0.001 mg/kg to about 1.5 mg/kg and/or from about 0.01 mg/kg to about 3 mg/kg.

### Combinations

Compounds and pharmaceutical compositions of the invention may be used in combination with one or more other therapeutic, prophylactic, diagnostic, or imaging agents. By “in combination with,” it is not intended to imply that the agents must be administered at the same time and/or formulated for delivery together, although these methods of delivery are within the scope of the present disclosure. Compounds and pharmaceutical compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of pharmaceutical, prophylactic, diagnostic, or imaging compositions in combination with agents that may improve their bioavailability, reduce and/or modify their metabolism, inhibit their excretion, and/or modify their distribution within the body.

### Dosing

The present invention provides methods comprising administering compounds of the invention to a subject in need thereof. These compounds may be administered to a subject using any amount and any route of administration effective for preventing, treating or diagnosing a disease, disorder, and/or condition (e.g., a disease, disorder, and/or condition relating to XLHED). The exact amount required will vary from subject to subject, depending on the species, age, and general condition of the subject, the severity of the disease, the particular composition, its mode of administration, its mode of activity, and the like. Compositions in accordance with the invention are typically formulated in dosage unit form for ease of administration and uniformity of dosage. It will be understood, however, that the total daily usage of the compositions of the present invention may be decided by the attending physician within the scope of sound medical judgment. The specific therapeutically or prophylactically effective dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

In certain embodiments, compositions in accordance with the present invention may be administered at dosage levels sufficient to deliver from about 0.0001 mg/kg to about 200 mg/kg, from about 0.001 mg/kg to about 0.01 mg/kg, from about 0.003 mg/kg to about 0.03 mg/kg, from about 0.005 mg/kg to about 0.05 mg/kg, from about 0.015 mg/kg to about 0.15 mg/kg, from about 0.02 mg/kg to about 0.2 mg/kg, from about 0.03 mg/kg to about 0.3 mg/kg, from about 0.05 mg/kg to about 0.5 mg/kg, from about 0.1 mg/kg to about 1 mg/kg, from about 0.15 mg/kg to about 1.5 mg/kg, from about 0.2 mg/kg to about 2 mg/kg, from about 0.3 mg/kg to about 3 mg/kg, from about 5 mg/kg to about 50 mg/kg, from about 10 mg/kg to about 60 mg/kg, from about 15 mg/kg to about 65 mg/kg, from about 20 mg/kg to about 70 mg/kg, or from about 30 mg/kg to about 80 mg/kg, from about 40 mg/kg to about 90 mg/kg, from about 50 mg/kg to about 100 mg/kg, from about 75 mg/kg to about 150 mg/kg, from about 100 mg/kg to about 150 mg/kg or at least 200 mg/kg of subject body weight per day, one or more times a day, to obtain the desired therapeutic, diagnostic or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, delivery comprises 5 administrations over a 2 week period.

In one embodiment, compounds and pharmaceutical compositions of the invention are administered using a split dose. As used herein, a “split dose” is the division of a single unit dose or total daily dose into two or more doses, e.g, two or more administrations of the single unit dose. As used herein, a “single unit dose” is a dose of any therapeutic administered in one dose/at one time/single route/single point of contact, i.e., single administration event. As used herein, a “total daily dose” is an amount given or prescribed in 24 hr period. It may be administered as a single unit dose.

In one embodiment, EDI200 will be administered using a suitable dose in the range of from about 0.0001 mg/kg to about 100 mg/kg, from about 0.001 mg/kg to about 0.01 mg/kg, from about 0.005 mg/kg to about 0.05 mg/kg, from about 0.02 mg/kg to about 0.2 mg/kg, from about 0.05 mg/kg to about 0.5 mg/kg, from about 0.1 mg/kg to about 1 mg/kg, from about 0.2 mg/kg to about 2 mg/kg, from about 5 mg/kg to about 50 mg/kg, from about 10 mg/kg to about 60 mg/kg, from about 20 mg/kg to about 70 mg/kg, or from about 30 mg/kg to about 80 mg/kg, from about 40 mg/kg to about 90 mg/kg, from about 50 mg/kg to about 100 mg/kg, from about 75 mg/kg to about 150 mg/kg, from about 100 mg/kg to about 150 mg/kg or at least 100 mg/kg of subject body weight per day.

The pharmaceutical composition may be administered once daily, or may be administered as two, three or more sub-doses at appropriate intervals throughout the day or even using continuous infusion or delivery through a controlled release formulation. In some embodiments, EDI200 contained in each sub-dose must be correspondingly smaller in order to achieve the total daily dosage. Dosing may also be according to multi-dosing schemes of one, two, three, four, five or more doses.

The dosing may administered as two, three or more sub-doses at appropriate intervals over a day, more than one day, week, 2 weeks, 3 weeks, 1 month or greater. The dosage unit may be administered using continuous infusion over an appropriate time interval or delivery may occur through a controlled release formulation. For example, EDI200 can be administered using continuous infusion over 1 minute, 5 minutes, 10 minutes, 15 minutes, 30 minutes, 45 minutes, 1 hour, 2 hours, 3 hours, 4 hours, 5 hours or more. The dosage unit can also be compounded for delivery over several days, e.g., using a conventional sustained release formulation which provides sustained release over a several day period. Sustained release formulations are well known in the art and are particularly useful for delivery of agents at a particular site, such as could be used with the agents of the present invention. In this embodiment, the dosage unit contains a corresponding multiple of the daily dose.

In one embodiment, EDI200 is administered twice weekly for 3 weeks.

The effect of a single dose on any particular phenotype or symptom can be long lasting, such that subsequent doses are administered at not more than 3, 4, or 5 day intervals, or at not more than 1, 2, 3, or 4 week intervals.

The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments. Estimates of effective dosages and in vivo half-lives for the individual pharmaceutical compositions encompassed by the invention can be made using conventional methodologies or on the basis of in vivo testing using an appropriate animal model.

In one embodiment, dosages of compounds of the invention are determined using animal models of ectodermal dysplasia alone or in connection with whole genome (or pathways specific such as for EDA and EDAR signaling) sequence analysis, whether DNA, RNA or protein, or combinations thereof. In some embodiments, tabby mice are treated with EDI200 and the effectiveness of the compound is tested using gene expression analysis Skin biopsies from such mice can be examined using quantitative PCR (qPCR) analysis for changes in transcript levels from EDA-A1-responsive genes. Using this model, EDI200 doses can be adjusted to achieve the desired expression level that correlates with therapeutic effectiveness. Similar analysis may be conducted in human patients receiving EDI200 treatment to determine whether dosages should be adjusted to achieve the desired upregulation of EDA receptor activity and resulting gene expression. In some embodiments

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous).

General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference).

### Coatings or Shells

Solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

### Properties of Pharmaceutical Compositions

The pharmaceutical compositions described herein can be characterized by one or more of bioavailability, therapeutic window and/or volume of distribution.

### Bioavailability

Compounds and pharmaceutical compositions of the invention, when formulated with one or more delivery agents and/or excipients as described herein, may exhibit an increase in bioavailability as compared compositions lacking delivery agents or excipients as described herein. As used herein, the term “bioavailability” refers to the systemic availability of a given amount of a compound of the invention administered to a mammal. Bioavailability can be assessed by measuring the area under the curve (AUC) or the maximum serum or plasma concentration (Cₘₐₓ) of the unchanged form of a compound following administration of the compound to a mammal. AUC is a determination of the area under the curve plotting the serum or plasma concentration of a compound along the ordinate (Y-axis) against time along the abscissa (X-axis). Generally, the AUC for a particular compound can be calculated using methods known to those of ordinary skill in the art and as described in G. S. Banker, Modern Pharmaceutics, Drugs and the Pharmaceutical Sciences, v. 72, Marcel Dekker, New York, Inc., 1996, herein incorporated by reference.

The Cₘₐₓ value is the maximum concentration of the compound achieved in the serum or plasma of a mammal following administration of the compound to the mammal. The Cₘₐₓ value of a particular compound can be measured using methods known to those of ordinary skill in the art. The phrases “increasing bioavailability” or “improving the pharmacokinetics,” as used herein mean that the systemic availability of a compound of the invention, measured as AUC, Cₘₐₓ, or Cₘᵢₙ in a mammal is greater, when co-administered with a delivery agent as described herein, than when such co-administration does not take place. In some embodiments, the bioavailability of a compound of the invention can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Therapeutic Window

Compounds and pharmaceutical compositions of the invention, when formulated into a composition with a delivery agent and/or excipient as described herein, can exhibit an increase in the therapeutic window of the administered composition as compared to the therapeutic window of the composition lacking a delivery agent or excipient as described herein. As used herein “therapeutic window” refers to the range of plasma concentrations, or the range of levels of therapeutically active substance at the site of action, with a high probability of eliciting a therapeutic effect. In some embodiments, the therapeutic window of compounds and pharmaceutical compositions of the invention when co-administered with a delivery agent as described herein can increase by at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or about 100%.

### Volume of Distribution

Compounds and pharmaceutical compositions of the invention, when formulated with one or more delivery agents and/or excipients as described herein, can exhibit an improved volume of distribution (V_{dist}), e.g., reduced or targeted, relative compositions lacking delivery agents or excipients as described herein. The volume of distribution (V_{dist}) relates the amount of active ingredient in the body to the concentration of active ingredient in the blood or plasma. As used herein, the term “volume of distribution” refers to the fluid volume that would be required to contain the total amount of active ingredient in the body at the same concentration as in the blood or plasma: V_{dist} equals the amount of active ingredient in the body/concentration of active ingredient in blood or plasma. For example, for a 10 mg dose and a plasma concentration of 10 mg/L, the volume of distribution would be 1 liter. The volume of distribution reflects the extent to which active ingredient is present in the extravascular tissue. A large volume of distribution reflects the tendency of a compound to bind to the tissue components compared with plasma protein binding. In a clinical setting, V_{dist} can be used to determine a loading dose to achieve a steady state concentration. In some embodiments, the volume of distribution of compounds and pharmaceutical compositions of the invention when co-administered with a delivery agent as described herein can decrease at least about 2%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%.

### Therapeutic Applications

Compounds and pharmaceutical compositions of the present invention may be used to stimulate, enhance or restore biochemical signaling between cells. In this manner, compounds of the present invention may be used to stimulate, enhance or restore cellular movement that relies on signaling between cells such as the movement of cells that occurs during different stages of development in multicellular organisms. Such development includes, but is not limited to cellular development, gastrulation, organogenesis, ectodermal development, mesodermal development, endodermal development, embryonic development, fetal development, prenatal development, antepartum development, perinatal development, neonatal development, infant development, toddler development, childhood development and adolescent development. In addition, compounds and pharmaceutical compositions of the invention may stimulate, enhance or restore cellular movements in human development, mouse development, rat development, dog development, primate development (including non-human primate development).

In one embodiment, compounds and pharmaceutical compositions of the invention may be used to correct defects in ectodermal development. The ectoderm is one of the primary germ layers that is formed in the early embryo. Differentiation of cells in the embryonic ectoderm leads to the formation of many of the outer tissues of the body including the skin (epidermis), tooth enamel and the lining of the mouth, nostrils, anus, hair, nails and sweat glands. Ectodermal differentiation also leads to the formation of components of the nervous system (such as the spine, brain and peripheral nerves).

Compounds and pharmaceutical compositions of the present invention may be used to inhibit or prevent disorders of the ectoderm, in particular ectodermal dysplasia. Such disorders characterized by ectodermal dysplasia include, but are not limited to Absence of Dermal Ridge Patterns, Onychodystrophy, and Palmoplantar Anhidrosis, Acrorenal-Ectodermal Dysplasia-Lipoatrophic Diabetes (AREDYLD) Syndrome, Agammaglobulinemia-Dwarfism-Ectodermal Dysplasia, Aggammaglobulinemia-Thymic Dysplasia-Ectodermal Dysplasia, Alopecia-Anosmia-Deafness-Hypogonadism, Alopecia-Onychodysplasia-Hypohidrosis, Alopecia-Onychodyaplasia-Hypohidrosis-Deafness, Alopecia Universalia-Onychodystrophy-Total Vitiligo, Amelocerebrohypohidrotic Syndrome, Ameloonychohypohidrotic Dysplasia, Ankyloblepharon-Ectodermal Defects-Cleft Lip and Palate (AEC) Syndrome, Anonychia With Bizarre Flexural Pigmentation, Arthrogryposis and Ectodermal Dysplasia, Baisch's Syndrome, Book's Dysplasia, Camarena Syndrome, Carey's Syndrome, Christ-Siemens-Tourains's (CST) Syndrome, Coffin-Siris's Syndrome, Congenital Insensitivity to Pain with Anhidrosis, Congenital Lymphedema, Hypoparathyroidism, Nephrotathy, Prolapsing Mitral Valve, and Brachytelephalangy, Cranioectodermal Syndrome, Curly Hair-Ankyloblepharon-Nail Dysplasia (CHANDS), Cystic Eyelids-Palmoplantar Keratosis-Hypodontia-Hypotrichosis, Dermotrichic Syndrome, Dermoodontodysplasia, Dyskeratosis Congenita, Ectodermal Defect With Skeletal Abnormalities, Ectodermal Dysplasia of the Head, Ectodermal Dysplasia With Palatal Paralysis, Ectodermal Dysplasia With Severe Mental Retardation, Ectodermal Dysplasia With Syndactyly, Ectodermal Dysplasia Syndrome With Tetramelic Deficiencies, Ectrodactyly-Ectodermal Dysplasia-Cleft Lip/Palate (EEC) Syndrome, Ellis-Van Creveld's Syndrome, Fischer-Jacobsen-Clouston's Syndrome, Fischer's Syndrome, Focal Dermal Hypoplasia (FDH) (Goltz) Syndrome, Fried's Tooth and Nail Syndrome, Gingival Fibromatosis and Hyperrtrichosis, Gingival Fibromatosis-Sparse Hair-Malposition of Teeth, Gorlin-Chaudhry-Moss' Syndrome, Growth Retardation-Alopecia-Pseudoanodontia-Optic Atrophy (GAPO), Hallermann-Streiffs Syndrome, Hairy Elbows Dysplasia, Hayden's Syndrome, Hypertrichosis and Dental Defects, Hypodontia and Nail Dysgenesis, Hypohidrotic Ectodermal Dysplasia-Autosomal Recessive, Hypohidrotic Ectodermal Dysplasia With Hypothyroidism, Hypohidrotic Ectodermal Dysplasia With Papillomas and Acanthosis Nigricans, Hypomelanosis of Ito, Ichthyosiform Erythroderma-Deafness-Keratitis, Incontinentia Pigmenti, Johanson-Blizzard's Syndrome, Jorgenson's Syndrome, Kirghizian Dermatoosteolysis, Lenz-Passarge's Dysplasia, Marshall's Syndrome I, Melanoleucoderma, Mesomelic Dwarfism-Skeletal Abnormalities-Ectodermal Dysplasia, Mikaelian's Syndrome, Naefeli-Franceschetti-Jadassohn's Dysplasia, Oculodentodigital (ODD) Syndrome I, Oculodentodigital (ODD) Syndrome II, Oculoosteocutaneous Syndrome, Odontoonychodermal Dysplasia, Odontoonychodysplasia, Odontoonychodysplasia With Alopecia, Odontoonychohypohidrotic Dysplasia With Midline Scalp Defect, Odontotrichomelic Syndrome, Onychotrichodysplasia With Neutropenia, Orofaciodigital (OFD) Syndrome I, Osteosclerosis and Ectodermal Dysplasia, Pachyonychia Congenita, Palmoplantar Hyperkeratosis and Alopecia, Papillon-Lefevre's Syndrome, Pili Torti and Enamel Hypoplasia, Pili Torti and Onychodysplasia, Rapp-Hodgkin's Syndrome, Regional Ectodermal Dysplasia With Total Bilateral Cleft, Robinson's Syndrome, Rosseli-Gulienetti's Syndrome, Rothmund-Thomsons's Syndrome, Sabinas Brittle Hair and Mental Deficiency Syndrome, Salamon's Syndrome, Schinzel-Giedion's Syndrome, Skeletal Anomalies-Ectodermal Dysplasia-Growth and Mental Retardation, Syndrome of Accelerated Skeletal Maturation, Failure to Thrive, and Peculiar Face, Trichodental Dysplasia, Trichodentoosseous (TDO) Syndrome I, Trichodentoosseous (TDO) Syndrome II, Trichodentoosseous (TDO) Syndrome III, Trichodysplasia-Onychogryposis-Hypohidrosis-Cataract, Trichofaciohypohidrotic Syndrome, Trichooculodermovertebral Syndrome, Trichoodontoonychial Dysplasia, Trichoodontoonychodermal Syndrome, Trichoodontoonychodysplasia With Pili Torti, Trichoodontoonycho-Hypohidrotic Dysplasia With Cataract, Trichoonychodental (TOD) Dysplasia, Trichoonychodysplasia With Xeroderma, Trichorhinophalangeal (TRP) Syndrome I, Triphalangeal Thumbs-Onychodystrophy-Deafness, Walbaum-Deheane-Schlemmer's Syndrome, Xeroderma-Talipes-Enamel Defect, X-linked Hypohidrotic Ectodermal Dysplasia (XLHED) and/or Zanier-Roubicek's Syndrome.

In a preferred embodiment, EDI200 is used to treat, reverse, ameliorate or prevent XLHED or the symptoms associated with XLEHD. Prenatal, neonatal, childhood, adolescent as well as adult treatments with EDI200 are contemplated.

### Alteration or Modification of Phenotypic Presentation

The present invention provides compounds and methods for the correction, alteration or mitigation of various phenotypic presentations associated with ectodermal dysplasia, specifically XLHED.

Phenotypic presentations of ectodermal dysplasia include, but are not limited to, hypodontia (characterized by missing or abnormally shaped teeth including, but not limited to, any of the first, second or third molars, or the first or second premolar, canine or first or second incisors, significant oligodontia, microdontia, conical tooth crowns, speech impairment due to tooth abnormalities and lack of enamel), hypohidrosis (characterized by the inability to perspire due to absent or sparse eccrine sweat glands, abnormal morphology or lack (or reduced number) of sweat glands, Meibomian glands, glands of the upper respiratory tract, sebaceous glands, salivary glands and other glands, the incapacity to regulate homeostatic body temperature in relation to environmental temperature, heat intolerance, recurrent fevers, hyperthermia, recurrent benign infections, increased susceptibility to bronchitis, pneumonia, ocular disease due to dry eyes, febrile seizures, brain damage and even death) and hypotrichosis (characterized by absent, sparse or abnormal morphology of the hair on the scalp, eyebrows and/or body, and alopecia). Phenotypic presentations also may include growth retardation, poor mastication and poor appearance.

Phenotypic presentations may be subcharacterized as disorders of the eyes such as absence of Meibomian glands, diminished lacrymal production, chronic keratitis sicca (dry conjunctiva) leading to corneal opacification if not treated; disorders of the nose such as, absence of sub-mucosal glands, oonosis (foul smell), frequent rhinitis resulting in antibiotic treatment; disorders of the respiratory tract such as absence of sub-mucosal glands, Increased mucous viscosity and a decreased mucous clearance (cystic fibrosis like syndrome), frequent broncho-pneumonia resulting in antibiotic treatment; disorders of the oral cavity such as conical teeth, reduced number of teeth, diminished numbers of salivary glands, mastication impairment, speech impairment, facial dysmorphia, low self-esteem, social impairment; disorders of the gastrointestinal tract, such as absence of sub-mucosal glands, increased mucous viscosity resulting in a decreased mucous clearance (in a cystic-fibrosis like syndrome); disorders of growth and size such as where growth and size may be compromised in infancy; disorders of the hair such as scarce thin hairs on the scalp; disorders of the skin such as absence of sebaceous glands, dry skin and atopic-like dermatitis, absence or reduced numbers of sweat glands, and incapacity to regulate body temperature.

In one embodiment, EDI200 is administered prior to the development of a given phenotype in an affected subject. In such an embodiment, administration of EDI200 may be preceded by the use of computer assisted screening technology to identify pre-symptomatic affected subjects. As a non-limiting example, infra-orbital crease or fold, fullness of paranasal tissue, low insertion columella, elongated face, sparse eyelashes, long chin, thin eyebrows, nasal tip overhang, wide, broad, prominent or high nasal bridge, vermillion lower lip eversion, lateral hypoplasia of eyebrows, depressed nasal bridge, short philtrum, prominent eyes, high anterior hairline, tall or wide forehead, and/or exaggerated cupid's bow measurements may be synthesized to create a score for the identification of affected subjects. In another embodiment, EDI200 compounds and pharmaceutical compositions of the present invention are administered to reduce or halt the development of a given phenotype in an affected subject. In another embodiment, EDI200 compounds and pharmaceutical compositions of the invention are administered to reverse the appearance of a given phenotype in an affected subject.

In one embodiment, EDI200 administration may activate signaling cascades, including but not limited to, NF-kappaB induction of sonic hedgehog (Shh) and Hedgehog signaling (Schmidt-Ullrich et al. Development (2006)133, 1045-1057). As a non-limiting example, activation or modification of signaling cascades results in the induction of effector gene expression, including but not limited to Shh, Ptch1, Ptch2, Glil, and EDAR, that directly alters the phenotype of an affected subject.

### Research Applications

Compounds and pharmaceutical compositions of the present invention may be used in research and scientific discovery. In one embodiment, EDI200 may be used in a research application where stimulation, activation, or enhancement of EDA receptor signaling is desired or necessary. In another embodiment, compounds of the invention may be used in conjunction with animal models of XLHED. In some embodiments, tabby mouse sebaceous gland gene expression analyses are used to evaluate efficacy.

In mice, the “Tabby” mouse was the first identified model of XLHED. This mouse is characterized by the spontaneous appearance of a sub-strain with abnormal hair and tooth development. Heterozygous females have a characteristic fur patterning similar to that of the tabby cat. These mice lack functional EDA protein due to a deletion mutation which results in a frame-shift resulting in the absence of the domain necessary for receptor binding and signaling that is critical for normal tooth, hair and sweat gland morphogenesis (Ferguson et al., 1997; Srivastava et al., 1997). Consequently, these mice have no sweat glands and no hair on the tail. The Tabby mouse currently is a widely used model for XLHED. In one embodiment, EDI200 may be used to reverse the phenotype of these mice through prenatal, neonatal and/or adult treatment. In a further embodiment, EDI200 may be useful as a treatment in this model while examining other disease parameters.

XLHED studies are also carried out in a dog model of the disease obtained through the crossing of a German shepherd strain identified with an XLHED phenotype (Casal et al., 2005) with a Beagle strain, more commonly used for laboratory experimentation. Beagles carrying the EDA mutation (splice site alteration) exhibit a phenotype equivalent in many significant respects to that of humans. In one embodiment, EDI200 may be used to reverse the phenotype of these dogs through prenatal, neonatal and/or adult treatment. In a further embodiment, EDI200 may be useful as a treatment in this model while examining other disease parameters.

In the human, the most common mutation associated with XLHED is a missense mutation.

### Manufacturing Process

The manufacturing process for pharmaceutical formulations, including EDI200 manufacture is described in Example 1 in more detail. The manufacturing process includes testing and controls to ensure the safety of the product. These tests and controls include, but are not limited to, testing of the Master Cell Bank (MCB), assessment of materials of biological origin, testing for viral, bacterial and/or mycoplasmal contaminants at the end of the cell culture process for each manufacturing batch, in-process controls through the cell culture and purification process, demonstration of retrovirus and MMV clearance, assessment of residual impurities and batch release testing.

Further the batches may also be characterized for additional physico-chemical attributes such as, but not limited to, glycosylation, sialylation, charge heterogeneity, primary structure heterogeneity, tertiary structure (confirmation of hexameric structure), residual impurity levels, and biological activity. As non-limiting examples, biological activity may be assessed using an in vitro cell-free assay such as the BIACORE™ (GE Healthcare Bio-Sciences, Sweden) binding assay or an in vitro Tabby mouse model that exhibits the phenotypic manifestations of XLHED.

The manufacturing process may also include release and stability testing to ensure identity, purity, biological activity and safety. Release testing may include, but is not limited to, visual appearance, concentration (e.g., by UV), pH, osmolality, size exclusion HPLC, SDS-PAGE, biological activity (e.g., in vitro Jurkat cell line-based assay where Jurkat cells express an EDAR-Fas fusion protein), bioburden, endotoxin, residual host cell protein, residual host cell DNA, particulate matter and sterility. Stability testing may include, but is not limited to, imaged capillary isoelectric focusing, long-term storage stability using accelerated temperatures.

### Kits

The pharmaceutical compositions and formulations of the invention may be packaged as a kit. Additionally, the kit may contain instructions for preparation and administration of the pharmaceutical composition and formulations.

### Kits for Therapeutic Use

The kit may be manufactured as a single use unit dose for one patient, multiple uses for a particular patient or the kit may contain multiple doses suitable for administration to multiple patients (“bulk packaging”). The kit components may be assembled in cartons, bottles, tubes and the like. Kits may also include instructions for administering the pharmaceutical compositions using any indication and/or dosing regimen described herein.

### Kits for Diagnostic Use

The kit may be manufactured for use as a diagnostic tool. The kit components may be assembled in cartons, bottles, tubes and the like. Kits may also include instructions for carrying out the desired diagnostic application. Kits of the present invention may be used to determine the level of biological and chemical compounds in mammalian bodily fluids and tissues using the techniques described herein.

In one embodiment, kits of the present invention can be used to detect EDI200 and/or anti-EDI200 antibodies in mammalian fluids and tissue. In a further embodiment, EDI200 and anti-EDI200 antibody levels can be detected in mammalian serum. Such kits could be useful for monitoring mammals undergoing treatments that include the use of EDI200, variants or fragments thereof. Such kits may include, but are not limited to colorimetric, radioactive, bioluminescent or fluorescent-based methods of detecting EDI200 or anti-EDI200 antibody levels. Additionally, diagnostic kits may be designed to carry out EDI200 and/or anti-EDI200 antibody detection according to the methods described in the examples herein.

### Definitions

For convenience, the meaning of certain terms and phrases employed in the specification, examples, and appended claims are provided below. The definitions are not meant to be limiting in nature and serve to provide a clearer understanding of certain aspects of the present invention.

The term “activation” as used herein refers to any alteration of a signaling pathway or biological response including, for example, increases above basal levels, restoration to basal levels from an inhibited state, and stimulation of the pathway above basal levels.

The term “biological sample” or “biologic sample” refers to a sample obtained from an organism (e.g., a human patient) or from components (e.g., cells) or from body fluids (e.g., blood, serum, sputum, urine, etc) of an organism. The sample may be of any biological tissue, organ, organ system or fluid. The sample may be a “clinical sample” which is a sample derived from a patient. Such samples include, but are not limited to, sputum, blood, blood cells (e.g., white cells), amniotic fluid, plasma, semen, bone marrow, and tissue or core, fine or punch needle biopsy samples, urine, peritoneal fluid, and pleural fluid, or cells therefrom. Biological samples may also include sections of tissues such as frozen sections taken for histological purposes. A biological sample may also be referred to as a “patient sample.” The term “cell type” refers to a cell from a given source (e.g., a tissue, organ) or a cell in a given state of differentiation, or a cell associated with a given pathology or genetic makeup.

As used herein, the term “compound” refers to a substance composed of two or more parts, components, elements or ingredients. In some embodiments, such components may include, but are not limited to atoms, molecules, macromolecules, amino acids, peptides, proteins, protein subunits, nucleic acids, lipids, sugars and combinations thereof. In one embodiment, compounds include EDI200 or variants thereof as described herein.

The term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

The term “correlate” or “correlation” as used herein refers to a relationship between two or more random variables or observed data values. A correlation may be statistical if, upon analysis by statistical means or tests, the relationship is found to satisfy the threshold of significance of the statistical test used.

As used herein, an “excipient” is a substance or composition that serves as the vehicle or medium for a drug or other active substance or composition.

The term “detectable” refers to an RNA expression pattern which is detectable via the standard techniques of polymerase chain reaction (PCR), reverse transcriptase-(RT) PCR, differential display, and Northern analyses, or any method which is well known to those of skill in the art. Similarly, protein expression patterns may be “detected” via standard techniques such as Western blots.

“Mammal” for purposes of treatment refers to any animal classified as a mammal, including humans, domestic and farm animals, and zoo, sports, or pet animals, such as dogs, horses, cats, cows, monkeys etc. Preferably, the mammal is a human.

The phrase “a method of treating” or its equivalent, when applied to, for example, XLHED refers to a procedure or course of action that is designed to reduce, eliminate or alter the phenotypic presentation and/or side effects associated with a disease or condition in an individual, or to alleviate the symptoms of said disease or condition. “A method of treating” a disease or disorder does not necessarily mean that the disease or disorder will, in fact, be completely eliminated, or that the symptoms of the disease or disorder will, in fact, be completely alleviated. Often, a method of treating cancer will be performed even with a low likelihood of success, but which, given the medical history and estimated survival expectancy of an individual, is nevertheless deemed an overall beneficial course of action.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

As used herein, the term “pharmaceutical composition” refers to a substance composed of two or more components useful in the treatment, cure, prevention or medical diagnosis of one or more diseases or disorders. In one embodiment, pharmaceutical compositions comprise EDI200 and one or more excipients. In some embodiments, pharmaceutical compositions comprise a sterile solution (pH 7.2) for intravenous infusion further comprising 5.0 mg/ml EDI200, 20 mM sodium phosphate, 300 mM sodium chloride and 0.02% Polysorbate 20.

The term “phenotypic presentation” refers to the macroscopic presentation of a disease. In one embodiment, the disease may be ectodermal dysplasia. Phenotypic presentations associated with ectodermal dysplasia include missing teeth, abnormally shaped teeth, abnormal morphology or lack (or reduced number) of sweat glands, lack of Meibomian glands, lack of glands of the upper respiratory tract, lack of sebaceous glands, lack of salivary glands, lack or abnormal morphology of various types of hair and/or alopecia.

The term “predicting” means a statement or claim that a particular event will, or is very likely to, occur in the future.

The term “prognosing” means a statement or claim that a particular biologic event will, or is very likely to, occur in the future.

The term “progression” or “disease progression” means the advancement or worsening of or toward a disease or condition.

The term “subject” refers to patients of human or other vertebrates in particular mammal and includes any individual it is desired to examine or treat using the methods according to the present invention. However, it will be understood that “patient” does not automatically imply that symptoms or diseases are present. As used herein, the term “patient” preferably refers to a human in need of treatment.

The term “treating” as used herein, unless otherwise indicated, means reversing, alleviating, inhibiting the progress of, or preventing, either partially or completely, the phenotypic (or otherwise, including genotypic) manifestations of a disease or condition. The term “treatment” as used herein, unless otherwise indicated, refers to the act of treating.

The term “treatment outcome” means the result of one or more treatments. Treatment outcomes may be positive or negative. The nature of the treatment outcome, such as a “positive” outcome may be objectively or subjectively measured. For example, a positive outcome may be reflected in the subjective characterization of the patient of their condition (e.g., they “feel” better), or it may be represented by an objective measurement of the disorder (e.g., an increase in hair growth, tooth morphology or ability to sweat).

The term “therapeutically effective agent” refers to compounds or pharmaceutical compositions that will elicit the biological or medical response of a tissue, organ, system, organism, animal or human that is being sought by the researcher, veterinarian, medical doctor or other clinician.

The term “therapeutically effective amount” or “effective amount” means the amount of the subject compound or combination that will elicit the biological or medical response of a tissue, organ, system, organism, animal or human that is being sought by the researcher, veterinarian, medical doctor or other clinician. In this context, a biological or medical response includes treatment outcomes.

The term ‘asymptomatic’ refers to individuals who have a disease or genetic disposition without any of the phenotypic outward symptoms of that same disease or genetic disposition.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### EXAMPLES

### Example 1

### EDI200 Manufacture and Characterization

Expression Vector p449 Construction

The EDI200 expression vector p449 was derived from the ligation of two plasmid fragments, one derived from PS1938 containing the EDI200 gene sequence and the other derived from the Invitrogen plasmid pEF1/myc-HisB.

Plasmid PS1938 containing the EDI200 gene sequences is described in Swee et al., (Swee L K, Ingold-Salamin K, Tardivel A, Willen L, Gaide O, Favre M, Demotz S, Mikkola M, Schneider P. (2009). J. Biol. Chem. 284: 27567-27576) and has the following EDI200 gene sequence components: (1) Gene sequence encoding for the signal sequence of the hemagglutinin protein of Influenza A virus (Swissprot accession number P03450; amino acids 1-15) (this gene sequence is for protein secretion and is not in the final EDI200 protein); (2) Gene sequence encoding for the human IgG1 Fc protein (Swissprot accession number P01857; amino acids 105-330); and (3) Gene sequence encoding for part of the extra-cellular domain of the EDA-A1 protein (Swissprot accession number Q92838; amino acids 238-391) and containing the entire TNF-homology domain of EDA-A1, but not its collagen domain.

The plasmid fragment containing the EDI200 gene was isolated from plasmid PS1938 after PCR amplification using the primers AX06 (5′-ATTTAGGTGACACTATAG-3′; SEQ ID NO. 2) and AX115 (5′-TCCAGTGTGGTGGAATTCATGGCTATCATCTACCTC-3′; SEQ ID NO. 3).

In addition to generating the EDI200 gene amplicon, the amplification introduced a 5′ EcoRI site. The PCR amplicon containing the EDI200 gene was then digested with EcoRI and NotI and then purified by agarose gel electrophoresis.

Plasmid pEF1/myc-HisB was digested with EcoRI and NotI to linearize and the resulting plasmid fragment was purified by agarose gel electrophoresis. The resulting fragment (6141 bp) and the EDI200 gene-containing PCR amplicon (1194 bp) were ligated and transformed into TOP10 E. coli (Invitrogen). The DNA from mini-preps derived from four separate colonies was extracted using a Nucleospin® plasmid kit (Clontech Laboratories) and the entire EDI200 gene was sequenced in both directions using the primers AX5 (5′-TAATACGACTCACTATAGGG-3′; Forward Primer for Nucleotides 1704-2702; SEQ ID NO. 4), AX116 (5′-CCGACGGCTCCTTCTTCC-3′; Forward Primer for Nucleotides 2376-3367; SEQ ID NO. 5), AX117 (5′-GGAAGAAGGAGCCGTCGG-3′; Reverse Primer for Nucleotides 1325-2320; SEQ ID NO. 6) and AX126 (5′-AGGCACAGTCGAGGCTGA-3′; Reverse Primer for Nucleotides 2043-3033; SEQ ID NO. 7).

To cover the sequence of the entire EDI200 gene on both DNA strands, two primers for the forward sequencing (AX5 and AX116) and two primers for the reverse sequencing (AX117 and AX126) were used. The forward and reverse sequencing covered the entire EDI200 gene, which is located within the nucleotides 1754 to 2941.

Based on the sequence information, one of the plasmid mini-preps was chosen and re-named plasmid p449. The EDI200 gene in this plasmid had the expected DNA sequence and the plasmid was used for construction of the EDI200 expression cell line.

The sequence of the expression plasmid p449 is given in SEQ ID NO. 8. The plasmid is 7336 base pairs in size.

Plasmid elements include the EDI200 gene under the control of the EF-1α promoter and the neomycin resistance gene under control of the SV40 promoter (used for selection). As described above, the plasmid sequence was confirmed from by position 1325 to 3367 which includes the entire EDI200 gene (located at position 1754 to 2941) as well as a portion of the EF-1α promoter and the entire BGH polyadenylation sequence.

### Cell Culture

The EDI200 drug substance is manufactured at the 500 L scale using cell culture methods employing a recombinant Chinese Hamster Ovary cell line in a 100 L single use bioreactor (SUB).

The CHO-S cell line (Invitrogen) was cultured in chemically defined medium (CD-CHO medium containing HT supplement (provides hypoxanthine and thymidine) and glutamine). All culturing was carried out at 37° C. in a 5% CO₂ incubator. The cell line was transfected with the EDI200 expression plasmid p449 in 24 well plates containing Opti-MEM and DMRIE-C. After incubation and washing steps using Opti-MEM and CHO-S, Geneticin® was added (on day 2) for selective pressure and growth in T25 flasks. After 10 days of incubation the cultures were moved to 5×96 well plates and wells were seeded at 1000 cells/well.

After 2 weeks of further selection and growth, the supernatants of individual wells were tested for product titer by protein A-based ELISA. The top 5 clones were selected and expanded. Limiting dilution subcloning was performed at 0.3 and 3 cells/well. After growth of the subclones, analysis of the clones by ELISA resulted in the selection of a high titer subclone. A small amount of the subclone was prepared and tested at Charles River Laboratories, (Malvern, Pa.). Testing results showed the cell bank was free of adventitious agents including microbial, mycoplasmal, and viral contaminants. In addition, the identity of the cell line as CHO-derived was confirmed.

The CHO culture was harvested by depth filtration and then purified through a series of column chromatography steps including protein A affinity column (MAb Select SuRe resin), ceramic hydroxyapatite (CHT type 1), cation exchange column chromatography (SP Sepharose HP), and anion exchange column membrane (Mustang Q membrane filter).

After purification, the culture underwent a low pH step for virus inactivation which further includes a Planova 20N virus removal filter. Ultrafiltration/diafiltration was performed to concentrate and diafilter (with a 30 kDa molecular weight cut off) the product into the final phosphate-based buffer where the polysorbate 20 was added. The solution was then filtered using a 0.2 μm filter before being bottled and stored at a temperature less than or equal to −65° C. EDI200 Characterization

The EDI200 drug substance was characterized using a variety of physico-chemical methods. The results provided confirmation of the primary structure of the EDI200 monomer and confirmation of the EDI200 hexameric tertiary structure. Primary structure heterogeneity as well as secondary structure (disulfide mapping) and post translational modifications including glycosylation structure and site occupancy were also assessed.

In the EDI200 hexamer, two monomeric species are connected by inter-chain disulfide bonds in the Fc region of the molecule and the hexameric structure is formed by association of three inter-chain disulfide linked dimers by non-covalent interactions. There are nine cysteine residues, with six forming three intra-disulfide linkages (Cys₄₀-Cys100, Cys₁₄₆-Cys₂₀₄, and Cys₃₂₁-Cys₃₃₅). Two cysteine residues form inter-disulfide linkages (Cys₅ and Cys₈) and one residue is unpaired (Cys₃₄₁).

The EDI200 monomer is glycosylated at Asn76, and Asn302 of SEQ ID NO. 1, however, four potential N-linked glycosylation sites are present at Asn76, Asn302, Asn333, and Asn361. Site occupancy of glycans was determined by LC/MS of tryptic peptides for Asn₇₆, and of chymotryptic peptides for Asn₃₀₂. Peptides were identified by the observed mass of the glycopeptide compared to the theoretical mass (with a limit of 15 ppm mass accuracy). Glycan structures were identified using mass; therefore glycan isomers were not distinguished.

The results show that the two sites differ in N-glycan structure and site occupancy. The Asn₇₆ is highly occupied, as shown by the relatively low levels of aglycosylation. The most abundant N-glycan structures at this site are bi-antennary glycan structures with a core fucose and terminating in 0, 1, or 2 galactose residues. None of the abundant glycans at this site were sialylated. The Asn₃₀₂ site is also highly occupied. The most abundant N-glycan structures at this site are tetra-antennary structures with core fucose and variable levels of sialylation. The overall sialic acid content in the EDI200 drug substance was approximately 0.7 pMol sialic acid/mol of EDI200 monomer. The predominant form observed is N acetylneuraminic acid with trace levels of N-glycolylneuraminic acid. 0.7 μmol sialic acid/pmol of EDI200.

The predicted molecular mass of reduced, deglycosylated, EDI200 monomer is 42498.2 Da based on the theoretical amino acid sequence. The molecular mass has been confirmed by LC/MS ESI-TOF as 42498.4 Da which agrees well with the predicted molecular mass. The approximate molecular mass of hexameric, glycosylated EDI200 has been determined using size exclusion chromatograph-multiangle laser light scattering (SEC-MALS). The molecular weight obtained (approximately 290,000 Da) is consistent with a hexameric structure consisting of six glycosylated monomers. While EDI200 exhibits charge heterogeneity, the major species has an isolectric point (pI) of approximately 7.4.

The final drug substance is supplied at a target product concentration of 10.0 mg/mL in 20 mM sodium phosphate, 300 mM sodium chloride, pH 7.2, 0.02% Polysorbate 20.

The EDI drug product is supplied at a target concentration of 5.0 mg/mL EDI200 in 20 mM sodium phosphate, 300 mM sodium chloride, pH 7.2, 0.02% Polysorbate 20 (TWEEN®20).

The EDI200 sterile solution is a clear to slightly opaque, essentially colorless sterile parenteral solution with a pH of 7.2. At the target concentration, each milliliter of the sterile solution contains approximately 5.0 mg of EDI200. The drug product is supplied as a frozen sterile liquid in 3 mL, 13 mm neck USP Type 1 borosilicate vials with a 13 mm gray butyl stopper (with FluroTec® on the plug only) and sealed with a 13 mm blue aluminum controlled score flip-off seal. EDI200 sterile solution for intravenous infusion is stored at −60 to −90° C. Prior to use as stipulated in the clinical protocol, the product is thawed at room temperature or under refrigerated conditions.

### Confirmation of Biological Activity

Biological activity was measured using an in vitro Jurkat cell-based method known in the art. The cell line used in the method is a Jurkat cell line, designated JOM2-2199 CL23sc20 (Lot SCL-20), which was transduced with a chimeric protein comprised of the extracellular domain of EDAR and the intracellular domain of Fas. This line was subcloned and a working cell bank prepared.

To initiate the assay, a vial of the working cell bank was cultured at 37° C. in a CO₂ incubator in Growth Medium (RPMI +9% fetal bovine serum). When sufficient cell mass was available, the cells were centrifuged and resuspended, and the cell suspension was added to 96-well microtiter plate wells containing a prepared reference standard and test article. The reference standard and test article are prepared by first diluting to a product concentration of 2700 ng/mL in growth medium and then further diluted to achieve three fold dilutions across a 96 well microtiter plate. Each concentration for the reference standard and for the test article are loaded onto the plate in triplicate prior to addition of cell suspension.

After the cell suspension and additional growth medium were added, the plate was incubated at 37° C. for 18-24 hours. Without wishing to be bound by theory, it is believed that during the incubation, EDI200 engages with the EDAR portion of the EDAR-Fas chimera and induces the apoptotic cascade, causing cell death in a concentration dependent manner.

The extent of remaining viable cells was measured by the addition of CellTiter 96® Aqueous One Solution (Promega). This solution contains a tetrazolium compound (3-(4,5-dimethylthiazol-2-yl)-5-(3-carboxymethoxyphenyl)-2-(4-sulfophenyl)-2H-tetrazolium, inner salt, also referred to as MTS) and an electron coupling reagent (phenazine ethosulfate (PES)). PES combines with MTS to form a stable solution according to the manufacturer. After addition of the MTS/PES solution the plates were incubated for an additional 7-8 hr and then the corrected absorbance (A₄₉₀-A₆₅₀) was recorded in a 96-well plate reader. The quantity of formazan product as measured by the amount of 490 nm absorbance correlates to the number of living cells in culture.

For each sample or set of samples, an internal control plate was prepared using the reference standard as the test article to provide assurance of acceptable performance of the cell line on a given day. Data analysis for the internal control plate and the sample plates was performed by plotting the mean and corrected absorbance values of the reference standard and test article against the final EDI200 concentration on a logio scale. The dose response curves were fitted using a 4-parameter logistic curve model. System suitability and sample acceptance criteria were analyzed using SoftMax Pro data and PLA 2.0 is used for parallelism evaluation. Testing for outliers was performed for each triplicate reading using a Dixon's Q-test. System suitability requirements have been established for acceptance of the assay for the standard curve on each plate, for the internal control plate, and also for the test article including: a) r² must be ≧0.950; b) requirements for precision results for each triplicate determination; c) the change in optical density (OD) response must be ≧0.25; d) the internal standard control must return a relative potency of ≧0.5 and ≦1.5; e) pass test for parallelism. If the above criteria are met, the test results are accepted and the relative potency of the sample is calculated as:

Relative Potency (%)=100×(EC₅₀ Reference Standard/EC₅₀ Test Article), where: EC₅₀ Reference Standard=SoftMax Pro calculated C-parameter from the standard curve. The EC₅₀ Test Article=SoftMax Pro calculated C-parameter from the test article curve. In this assay, reference standards are prepared using a bench scale process and the composition was identical to EDI200 drug substance composition.

### ELISA Assay

Biological activity may be determined using an enzyme linked immunosorbent assay (ELISA).

### Example 2

### EDI200 in Non Human Primates: Detection in Serum

A quantitative pharmacokinetic ligand binding method for the measurement of EDI200 was developed and validated for use with non-human primate (NHP) serum under GLP regulations. The assay was found to perform well within the expected industry standards and was validated to have 42 days of stability. The assay was found to be specific for EDI200 and demonstrated acceptable accuracy, precision, 4.5 hour short term stability at ambient conditions, three cycle freeze/thaw stability at −10 to −30° C. and −50 to −90° C., and 42 day long term storage stability at −10 to −30° C. and −50 to −90° C. Furthermore, the assay was determined to have a good range (3.91 to 250 ng/mL) and to be highly sensitive with a lower limit of quantification (LLOQ) of 3.91 ng/mL or 39.1 ng/mL in undiluted serum. Samples above the limit of quantitation can be diluted up to 1:160,000 within the range of the standard curve to obtain accurate results.

### Materials and Methods

Outlined in Tables 1 and 2 are the matrix and reagent information for the present study.

TABLE 1
Matrix information and preparation

| | |
|---|---|
| Matrix | Non-human primate serum |
| Anticoagulant/Stabilizer | None |
| Source | Bioreclamation Inc. |
| Pool Preparation | 10 male lots and 10 female lots; |
| | aliquots made as applicable |
| Pool Storage | Frozen at −10 to −30° C. |

TABLE 2
Reagent Information and Preparation

| | Capture | Detection | | | |
|---|---|---|---|---|---|
| Identity | Reagent | Reagent | Analyte | EDA-A1 | IgG₁ |
| Description | hEDAR-mFc | Mouse anti- | EDI200 | Recombinant | Recombinant |
| | | human IgG | | human | human IgG₁ |
| | | (Fc) CH2 | | Ectodysplasin | Fc (hFc) |
| | | Domain - | | A1 | |
| | | HRP | | | |
| Strength/ | 1 mg/mL | 1 mg/mL | 9.4 mg/mL | | |
| Purity | | | | | |
| Source | Edimer | AbD Serotec, | CMC ICOS | R&D System, | R&D System, |
| | Pharmaceuticals, | Oxford, UK | Biologics, | Minneapolis, | Minneapolis, |
| | Cambridge, MA | | Bothell, WA | MN | MN |
| Storage | Frozen at −50 | Refrigerated at | Frozen at −50 | | |
| | to −90° C. | 2 to 8° C. | to −90° C. | | |
| Dilution | 1 μg/mL in | 1:5000 in | 1 mg/mL in 20 | | |
| | 1X PBS for | assay buffer | mM Sodium | | |
| | plate coating | (0.1% BSA, | Phosphate, | | |
| | | 0.05% | 300 mM | | |
| | | TWEEN ®20 | NaCl, 0.02% | | |
| | | in 1X PBS) | TWEEN ®20, | | |
| | | | pH 7.2 | | |

### Analyte Preparation

EDI200 stock solution preparation was prepared by combining and mixing 330 μL of 9.4 mg/mL EDI200 and 2772 μL of 20 mM Sodium Phosphate, 300 mM NaCl, 0.02% TWEEN®20 (Sigma-Aldrich, St. Louis, Mo.) and adjusting the pH to 7.2. Single use aliquots were prepared, assigned the expiration date of the EDI200 lot from which they were prepared and stored at −70° C. until use.

### Buffer and Solution Preparation

200 mM sodium phosphate dibasic heptahydrate was prepared by combining and mixing 5.36 g of sodium phosphate dibasic heptahydrate (Sigma Aldrich, St. Louis, Mo.) and 100 mL ultrapure deionized water. This reagent was stored at room temperature and used within 3 months from the date of preparation.

200 mM sodium phosphate monobasic was prepared by combining and mixing 2.4 g of sodium phosphate monobasic (Sigma Aldrich, St. Louis, Mo.) and 100 mL ultrapure deionized water. This reagent was stored at room temperature and used within 3 months from the date of preparation.

100 mM sodium phosphate buffer was prepared by combining and mixing 33 mL of 200 mM Sodium Phosphate Dibasic Heptahydrate, 17 mL of 200 mM Sodium Phosphate Monobasic and 50mL ultrapure deionized water. This reagent was stored at room temperature and used within 3 months from the date of preparation.

Reagent buffer (20 mM sodium phosphate, 300 mM NaCl and 0.02% TWEEN®20, pH 7.2) was prepared by combining and mixing 50 mL of 100 mM Sodium Phosphate Buffer, 4.383 g of NaCl (Sigma Aldrich, St. Louis, Mo.), 50 μL TWEEN®20 (Sigma Aldrich, St. Louis, Mo.) and 200 mL of ultrapure deionized water. The pH was then adjusted to 7.2±0.05 and filtered through a 0.22 μm CA filter unit (Corning, Corning, N.Y.). This reagent was stored at room temperature and used within 3 months from the date of preparation.

10× phosphate buffered saline (PBS) was prepared by combining and mixing one pack of PBS 10× Ready Concentrate (Fisher Scientific, Pittsburgh, Pa.) with 1 L deionized water. This reagent was stored at room temperature and used within 3 months from the date of preparation.

1× PBS was prepared by combining and mixing 100 mL 10× PBS and 900 mL deionized water. The solution was filtered using a 0.22 μm CA filter (Corning, Corning, N.Y.), stored at room temperature and used within 3 months from the date of preparation.

1× PBST (0.05% TWEEN®20 in 1× PBS) was prepared by combining and mixing 10 mL TWEEN®20 and 2 L 10× PBS. The solution was brought to a final volume of 20 L with deionized water, stored at room temperature and used within 3 months from the date of preparation.

Blocking buffer (3% BSA in 1× PBS) was prepared by combining and mixing 15 g of BSA (Sigma Aldrich, Cat# A7030) and 500 mL of 1× PBS. The solution was filtered using a 0.22 μm CA filter unit (Corning, Cat# 430513), stored at 2-8° C. and used within 1 month of preparation.

### Serum Handling and Preparation

Non human primate (NHP) serum was prepared by combining and mixing equal amounts of normal Cynomolgus Macaque serum lots from a commercial source. Aliquots were store at −20° C.

### Preparation of Assay Components

Coated plates prepared according to the following protocol. 1 μg/mL hEDARmFc coating solution was prepared in 1× PBS (e.g., 11 μL of 1 mg/mL hEDARmFc was combined with 11 mL 1× PBS). 100 μL of the 1 μg/mL hEDARmFc coating solution was added to each well of the assay plates. Plates were then sealed and incubated at 2-8° C. for 12-24 hours. Next, plates were washed 5 times with 300 μL 1× PBST per well and tapped on absorbent material to remove residual liquid. 150 μL of Blocking Buffer (3% BSA in 1× PBS) was added to each well before sealing and incubating at room temperature for 120±10 minutes. Next, plates were washed 5 times with 300 μL 1× PBST per well and tapped on absorbent material to remove residual liquid. Finally, plates were sealed and stored at 2-8° C. until use (not more than 1 week in storage)

Assay buffer (0.1% BSA, 0.05% TWEEN®20 in 1× PBS) was prepared by combining and mixing 1 g of BSA (Sigma Aldrich, St. Louis, Mo.) with 1000 mL 1× PBS and 500 μL of TWEEN®20. The solution was filtered using a 0.22 μm CA filter unit (Corning, Corning, N.Y.), stored at 2-8° C. and used within 1 month of preparation.

Stop Solution (˜2N Sulfuric Acid) was prepared by combining and mixing 20 mL of 35N Sulfuric Acid (Fisher, Pittsburgh, Pa.) and 330 mL deionized water. This reagent was stored at room temperature and used within 1 year of the date of preparation. Assay buffer with 10% serum was prepared by combining and mixing 4004 NHP Serum and 3.6mL of Assay Buffer.

Standards and quality controls were prepared for the assay. 25 μg/mL EDI200 Dilution 1 was prepared by diluting 1 mg/mL EDI200 stock in NHP Serum (e.g., 5 μL 1 mg/mL EDI200 stock was combined with 195 μL NHP Serum). 2.5 μg/mL EDI200 Dilution 2 was prepared by diluting 25 μg/mL EDI200 Dilution 1 in NHP Serum (e.g., 30 μL EDI200 Dilution 1 was combined with 270 μL NHP Serum).

Quality controls (QCs) were prepared in tubes according to Table 3 and assay standards were prepared in tubes according to Table 4.

TABLE 3
Preparation of quality controls (QCs)

| Stan- | | | Final |
|---|---|---|---|
| dard/ | | | Concentra- |
| Tube | Transfer | Diluent | tion |
| 10X QC | 80 μL of 2.5 μg/mL | 120 μL NHP | 10000 ng/mL |
| High | EDI200 Dilution 2 | Serum | |
| 10X QC | 50 μL of 10X QC High | 50 μL NHP | 500 ng/mL |
| Mid | | Serum | |
| 10X QC | 25 μL of 10X QC Mid | 100 μL NHP | 100 ng/mL |
| Low | | Serum | |
| 1X QC | 40 μL 10X QC High | 360 μL Assay | 100 ng/mL |
| High | | Buffer | |
| 1X QC | 40 μL 10X QC Mid | 360 μL Assay | 50 ng/mL |
| Mid | | Buffer | |
| 1X QC | 40 μL 10X QC Low | 360 μL Assay | 10 ng/mL |
| Low | | Buffer | |

TABLE 4
Preparation of Assay Standards

| Stan- | | | Final |
|---|---|---|---|
| dard/ | | | Concentra- |
| Tube | Transfer | Diluent | tion |
| 1 | 100 μL of 2.5 μg/mL | 900 μL of Assay Buffer | 250 ng/mL |
| | EDI200 | | (ULOQ) |
| 2 | 400 μL of Tube 1 | 400 μL of Assay Buffer | 125 ng/mL |
| | | containing 10% Serum | |
| 3 | 400 μL of Tube 2 | 400 μL of Assay Buffer | 62.5 ng/mL |
| | | containing 10% Serum | |
| 4 | 400 μL of Tube 3 | 400 μL of Assay Buffer | 31.25 ng/mL |
| | | containing 10% Serum | |
| 5 | 400 μL of Tube 4 | 400 μL of Assay Buffer | 15.63 ng/mL |
| | | containing 10% Serum | |
| 6 | 400 μL of Tube 5 | 400 μL of Assay Buffer | 7.81 ng/mL |
| | | containing 10% Serum | |
| 7 | 400 μL of Tube 6 | 400 μL of Assay Buffer | 3.91 ng/mL |
| | | containing 10% Serum | (LLOQ) |
| 8 | 400 μL of Tube 7 | 400 μL of Assay Buffer | 1.95 ng/mL |
| | | containing 10% Serum | |
| Blank | NA | 400 μL of Assay Buffer | 0 ng/mL |
| | | containing 10% Serum | |

### Assay Procedure

For validation, ELISA plates (96-well Microtiter Plates, Nunc 96F Maxisorp (Thermo Fisher, Pittsburgh, Pa.)) were coated with 1 μg/mL hEDARmFc coating solution. For the binding step, standards, QCs, Blank (assay buffer alone), and other required samples were added to the appropriate wells of an assay plate. Plates were incubated for approximately 120 minutes and then washed. For the detection step, the detection reagent was added to each required well and the plate was incubated for approximately 60 minutes and then washed. For the substrate step, Ultra TMB (1-Step Ultra TMB-ELISA Substrate (Thermo Scientific, Pittsburgh, Pa.)) was added to each well and the plate was incubated for approximately 20 to 30 minutes. Color development was monitored at 650 nm so that development could be discontinued when the OD of the 250 ng/mL standard was between 1.3 and 1.4. To stop the color reaction, 2N sulfuric acid was added to each well. The OD at 450 nm was determined with the correction wavelength set at 540 nm.

Sample concentrations were calculated using a standard curve constructed from the standards' concentrations and absorbance values. A 4 Parameter logistic (4PL) curve fit was used.

### Results and Discussion

### Accuracy and Precision

For core validation and preliminary stability batches, two sets of QC low, QC mid, and QC high samples were run in triplicate wells per level on the plate. For all other batches, one set of QC samples were run in triplicate wells on the plate. Accuracy and precision of the method were analyzed over three batches (batches 2, 3, and 4), across 2 days, by two separate analysts for a total of 18 results per QC level. Intra-assay accuracy and precision, reflective of the variation of the same preparation across replicates, was calculated for each QC level within each independent batch. Inter-assay accuracy and precision, reflective of the variation of multiple preparations of the same QC levels, was calculated from the values obtained across the three core validation batches. The assay meets criteria for both intra- and inter-assay accuracy and precision. Single individual wells for QC low and QC high in batch 4 had an absolute mean bias (% RE) outside ±20% and were excluded from statistics. The intra-assay and inter-assay accuracy and precision results are presented in Table 5. % CV stands for coefficient of variation and % RE stands for percent relative error.

TABLE 5
Accuracy and precision results

| | QC Low (10 ng/mL) | | | QC Mid (50 ng/mL) | | | QC High (100 ng/mL) | | |
|---|---|---|---|---|---|---|---|---|---|
| | Mean | % | % | Mean | % | % | Mean | % | % |
| Batch | Conc. | CV | RE | Conc. | CV | RE | Conc. | CV | RE |
| 2 | 11.6 | 4 | 16 | 48.7 | 3 | −3 | 104 | 3 | 4 |
| 3 | 10.2 | 2 | 2 | 49.6 | 4 | −1 | 102 | 2 | 2 |
| 4 | 10.5 | 6 | 5 | 51.2 | 1 | 2 | 108 | 8 | 8 |

Inter-assay statistics

| | | | | | | | | |
|---|---|---|---|---|---|---|---|---|
| 10.8 | 7 | 8 | 49.8 | 3 | 0 | 105 | 5 | 5 |

### Dilutional Linearity

Due to the large range of serum concentrations expected during sample analysis, dilutional linearity was performed as two independent sets within a single run. For the first set, a 22,000 ng/mL sample was prepared in undiluted serum and then diluted to 2200 ng/mL using assay buffer. This sample underwent serial dilutions using the assay buffer containing 10% serum to obtain five dilution levels within the range of the curve (4.4 to 220 ng/mL) and two dilution levels outside the range of the curve. For the second set, a 1000 μg/mL sample was prepared in undiluted serum and then diluted to 100 μg/mL using assay buffer. This sample underwent serial dilutions using the assay buffer containing 10% serum to obtain five dilution levels within the range of the curve (100 to 6.25 ng/mL) and two dilution levels outside the range of the curve. The dilutional linearity samples met acceptance criteria for accuracy and precision. These data demonstrate that samples above the limit of quantitation can be diluted up to 1:160,000 within the range of the standard curve and accurate results can be obtained. The preparation of the 2200 ng/mL and 100 μg/mL samples reflects immunology method requirements for analyzing unknown samples (all serum samples are initially diluted 1:10 using assay buffer). The dilutional linearity results are presented in Table 6.

TABLE 6
Dilutional linearity results

| | | | | |
|---|---|---|---|---|
| Nominal | Dilutional | Mean | | |
| Conc. | Level | Conc. | % CV | % RE |

Set 1

| | | | | |
|---|---|---|---|---|
| 440 | 1:50 | 205 | 4 | −53 |
| 220 | 1:100 | 239 | 4 | 8 |
| 44 | 1:500 | 48.1 | 0 | 9 |
| 22 | 1:1,000 | 23.7 | 2 | 8 |
| 11 | 1:2,000 | 13.2 | 1 | 20 |
| 4.4 | 1:5,000 | 4.98 | 5 | 13 |
| 2.2 | 1:10,000 | 1.7 | 8 | −23 |

Set 2

| | | | | |
|---|---|---|---|---|
| 500 | 1:2,000 | 199 | 4 | −60 |
| 100 | 1:10,000 | 99.1 | 2 | −1 |
| 50 | 1:20,000 | 48.8 | 1 | −2 |
| 25 | 1:40,000 | 27.4 | 2 | 10 |
| 12.5 | 1:80,000 | 14.2 | 3 | 14 |
| 6.25 | 1:160,000 | 6.79 | 1 | 9 |
| 3.1 | 1:320,000 | 4.01 | 19 | 29 |

### Lower Limit of Quantification (LLOQ) and Upper Limit of Quantification (ULOQ)

The estimated LLOQ and ULOQ were tested as six replicates per concentrations. The LLOQ and ULOQ samples met acceptance criteria for accuracy and precision. These data demonstrate that the LLOQ and ULOQ for this assay are 3.91 and 250 ng/mL, respectively. The LLOQ and ULOQ determination results are presented below in Table 7.

TABLE 7
LLOQ and ULOQ results

| | Nominal | Mean | | |
|---|---|---|---|---|
| | Conc. | Conc. | | |
| Sample | (ng/mL) | ng/mL | % CV | % RE |
| LLOQ | 3.91 | 3.57 | 16 | −9 |
| ULOQ | 250 | 251 | 5 | 1 |

### Selectivity

Method selectivity was assessed by evaluating assay responses to samples prepared using materials related to EDI200, including Recombinant Human IgG Fc (hFc), Recombinant Human EDA-A1 Ectodysplasin A1 (EDA-A1), and EDA-A1 with hFc. Initially, these solutions were prepared at 100 ng/mL, the same concentration as the QC high samples. The calculated results from these samples were to be below the concentration of the LLOQ for the method to be selective for EDI200. The initial results for 100 ng/mL EDA-A1 and 100 ng/mL EDA-A1+100 ng/mL hFc showed positive EDI200 response (>5 ng/mL). The method did not detect hFc alone.

Further selectivity testing was performed for EDA-A1 with and without hFc. For this additional testing, samples were prepared at the QC high concentration but serial 2-fold dilutions were performed until the resultant concentration was below the LLOQ and all samples were analyzed. Similar results were observed when EDA-A1 was tested in the presence and absence of hFc, indicating that the observed effect is due to the EDA-A1, not hFc. The data demonstrates that this was a concentration-specific result and samples spiked at ≦50 ng/mL were below the assay's limit of quantitation (BLQ). EDI200 contains the TNF homology domain of EDA-A1 that is fused to the Fc portion of human IgG1. The human recombinant EDA-A1 used in this assay is not fully representative of the EDA-A1 component of EDI200, as it contains both the collagen as well as the TNF homology domains (Ser160-Ser391). The results observed in the EDA-A1 spiked samples are likely due to non-specific binding interactions between the detection antibody and the recombinant human EDA-A1 molecule. Given the difference in protein source as well as protein sequence and structure, the selectivity results observed with recombinant human EDA-A1 cannot be directly correlated to the EDA-A1 TNF homology domain contained within EDI200. The selectivity results are presented below in Table 8. Concentrations are expressed as ng/mL.

TABLE 8
Selectivity results

| | Batch LLOQ | | hFc | | EDA-A1 | | EDA-A1/hFc | |
|---|---|---|---|---|---|---|---|---|
| | Mean | Mean | Mean | Mean | Mean | Mean | Mean | Mean |
| Batch | OD | Conc. | OD | Conc. | OD | Conc. | OD | Conc. |
| 5 | 0.245 | 3.82 | 0.116 | BLQ | 0.292 | 5.43 | 0.289 | 5.34 |
| | Batch LLOQ | | EDA-A1 | | | EDA-A1/hFc | | |
| | Mean | Mean | Nominal | Mean | Mean | Nominal | Mean | Mean |
| Batch | OD | Conc. | Conc. | OD | Conc. | Conc. | OD | Conc. |
| 7 | 0.25 | 3.89 | 100 | 0.316 | 6.14 | 100 | 0.275 | 4.77 |
| | | | 50 | 0.234 | BLQ | 50 | 0.214 | BLQ |
| | | | 25 | 0.18 | BLQ | 25 | 0.18 | BLQ |
| | | | 12.5 | 0.14 | BLQ | 12.5 | 0.15 | BLQ |
| | | | 6.25 | 0.119 | BLQ | 6.25 | 0.125 | BLQ |
| | | | 3.13 | 0.116 | BLQ | 3.13 | 0.121 | BLQ |

### Stability

Stability was assessed by comparing the data from QC preparations following applicable storage conditions to the nominal concentrations. In addition, each QC set must meet acceptance criteria for accuracy and precision. A set of aliquots were prepared from each of the two 10× QC low and QC high concentrations from batch 5. Short term stability testing was assessed by storing the two sets of 10× QC samples at ambient conditions for at least 4.5 hours, then analyzing fresh 1× dilutions (in assay buffer). The short term stability results are presented below in Table 9. Concentrations are expressed as ng/mL.

TABLE 9
Short term storage stability results

| | | QC Low (10 ng/mL) | | | QC High (100 ng/mL) | | |
|---|---|---|---|---|---|---|---|
| Storage | | Mean | % | % | Mean | % | % |
| Condition | Replicate | Conc. | CV | RE | Conc. | CV | RE |
| Ambient | A | 9.15 | 3 | −9 | 96.1 | 2 | −4 |
| | B | 9.56 | 3 | −4 | 99.3 | 4 | −1 |

Mean accuracy and precision of the short term stability samples were acceptable for the sample sets at each level. A single individual well for QC low set A had % RE outside ±20% and was excluded from statistics. Therefore, samples are considered stable for at least 4.5 hours when stored at ambient conditions.

### Freeze/Thaw Cycle Stability

Two sets of aliquots were prepared from each of the two 10× QC low and QC high concentrations from batch 4. One set was stored frozen at −10 to −30° C., and the second set was stored frozen at −50 to −90° C. Freeze/thaw stability testing was performed by thawing and re-freezing 10× QC samples up to three times. Following the third freeze/thaw cycle, fresh 1× dilutions were prepared and analyzed. The freeze/thaw cycle stability results are presented below in Table 10.

TABLE 10
Freeze/Thaw Cycle Stability Results

| | | QC Low (10 ng/mL) | | | QC High (100 ng/mL) | | |
|---|---|---|---|---|---|---|---|
| Frozen Cycle | | | % | % | | % | % |
| Condition | Replicate | Conc. | CV | RE | Conc. | CV | RE |
| −10 to −30° C. | A | 10.5 | 1 | 5 | 106 | 2 | 6 |
| | B | 10.9 | 1 | 9 | 110 | 2 | 10 |
| −50 to −90° C. | A | 10.9 | 1 | 9 | 115 | 0 | 15 |
| | B | 11.4 | 2 | 14 | 110 | 5 | 10 |

Mean accuracy and precision of the freeze/thaw cycle stability samples were acceptable for the sample sets at each level at −10 to −30° C. and −50 to −90° C. Therefore, samples are considered stable for up to three freeze/thaw cycles at both storage temperatures.

### Long Term Storage and Stability

Two sets of aliquots were prepared from each of the two 10× QC low and QC high concentrations from batch 4. One set was stored frozen at −10 to −30° C., and the second set was stored frozen at −50 to −90° C. After the required frozen storage intervals, fresh 1× dilutions were prepared and analyzed. The long term storage stability results are presented below in Table 11.

TABLE 11
Long term storage stability results

| | | QC Low (1:40,000) | | | QC High (1:10,000) | | |
|---|---|---|---|---|---|---|---|
| Storage | | | % | % | | % | % |
| Condition | Replicate | Conc. | CV | RE | Conc. | CV | RE |

7 day

| | | | | | | | |
|---|---|---|---|---|---|---|---|
| −10 to −30° C. | A | 11.1 | 1 | 11 | 115 | 3 | 15 |
| | B | 10.9 | 6 | 9 | 108 | 4 | 8 |
| −50 to −90° C. | A | 10.8 | 3 | 8 | 109 | 3 | 9 |
| | B | 11.3 | 1 | 13 | 105 | 1 | 5 |

28 day

| | | | | | | | |
|---|---|---|---|---|---|---|---|
| −10 to −30° C. | A | 10.3 | 2 | 3 | 95.0 | 4 | −5 |
| | B | 10.6 | 2 | 6 | 98.1 | 1 | −2 |
| −50 to −90° C. | A | 9.8 | 7 | −2 | 89.3 | 7 | −11 |
| | B | 10.7 | 5 | 7 | 91.7 | 2 | −8 |

42 day

| | | | | | | | |
|---|---|---|---|---|---|---|---|
| −10 to −30° C. | A | 11.0 | 5 | 10 | 104 | 3 | 4 |
| | B | 11.3 | 3 | 13 | 101 | 1 | 1 |
| −50 to −90° C. | A | 10.6 | 3 | 6 | 99.8 | 2 | 0 |
| | B | 10.7 | 1 | 7 | 96.7 | 3 | −3 |

Mean accuracy and precision of the long term storage stability samples were acceptable for the sample sets at each level at −10 to −30° C. and −50 to −90° C. at 7, 28, and 42 days. Therefore, samples are considered stable for up to 42 days at both storage temperatures.

### Conclusion

The assay performs well within the expected industry standards and is considered validated as having 42 days of stability. The assay was selective (specific) for EDI200 and demonstrated acceptable accuracy, precision, 4.5 hour short term stability at ambient conditions, three cycle freeze/thaw stability at −10 to −30° C. and −50 to −90° C., and 42 day long term storage stability at −10 to −30° C. and −50 to −90° C. Furthermore, the assay has a good range (3.91 to 250 ng/mL), and is highly sensitive (LLOQ of 3.91 ng/mL or 39.1 ng/mL in undiluted serum). Samples above the limit of quantitation can be diluted up to 1:160,000 within the range of the standard curve and accurate results can be obtained.

### Example 3

### Ligand Binding Method to Detect Anti-EDI200 Antibodies in Non Human Primate Serum

The present study was performed to develop a method to measure anti-EDI200 antibodies. The method was then validated for use with non-human primate (NHP) serum matrix.

### Materials and Methods

### Matrix and Reference Antibody Information and Preparation

The matrix used was from non-human primate (Cynomolgus monkey) serum and was procured from a commercial source (Bioreclamation Inc, Westbury, N.Y.). The reference antibody, hyperimmune anti-EDI200 serum, was created by immunizing a Tabby mouse with Fc-EDA1. At day 10, serum was collected and the mouse was boosted. At day 15 post-boost, serum was collected again. This serum was used as received. As needed, hyperimmune anti-EDI200 serum was added to NHP serum to yield a 1:1000 dilution of hyperimmune sera.

### Analyte Preparation

EDI200 stock solution (9.4 mg/mL, batch number 11-0015, CMC ICOS Biologics, Bothell, Wash.) was diluted to 1 mg/mL in buffer (pH 7.2) containing 20 mM Sodium Phosphate, 300 mM NaCL and 0.02% TWEEN®20 (Sigma-Aldrich, St. Louis, Mo.). Conjugation of EDI200 with biotin and MSD SULFO-TAG (Meso Scale Discovery, Gaithersburg, Md.) was carried out with 1 mg quantities of EDI200. An EDI200 Mastermix was prepared by combining Biotin-EDI200 and Sulfotag-EDI200 in assay buffer at a final concentration of 62.5 ng/mL for each.

### Additional Antibody Preparation

Donkey anti-human IgG (Jackson ImmunoResearch Laboratories, Inc, West Grove, Pa.) was used as a surrogate antibody for initial sensitivity testing. Mouse monoclonal (Renzo-1) to EDA (AbCam, Cambridge, Mass.) was used as a surrogate antibody for final sensitivity testing. Mouse anti-human CD106 (BD Biosciences, San Diego, Calif.) was used as an irrelevant antibody for specificity testing.

### Buffer Preparation

10× PBS was prepared by combining and mixing one pack PBS 10× Ready Concentrate (Fisher Scientific, Pittsburgh, Pa.) and 1 L deionized water. This reagent was stored at room temperature and used within 3 months from the date of preparation.

1× PBS preparation was prepared by combining and mixing 100 mL 10× PBS and 900 mL deionized water. The solution was filtered using a 0.22 μm CA filter (Corning Inc., Tewksbury, Mass.), stored at room temperature and used within 3 months from the date of preparation.

Blocking Buffer (3% BSA in 1× PBS) was prepared by combining and mixing 15 g of BSA (Sigma Aldrich, St. Louis, Mo.) and 500 mL of 1× PBS. The solution was filtered using a 0.22 μm CA filter unit (Corning Inc., Tewksbury, Mass.), stored at 2-8° C. and used within 1 month of preparation.

Assay Buffer (0.1% BSA, 0.05% TWEEN®20 in 1× PBS) was prepared by combining and mixing 1 g of BSA (Sigma Aldrich, St. Louis, Mo.), 1000 mL 1× PBS and 500 μL of TWEEN®20. The solution was filtered using a 0.22 μm CA filter unit (Corning Inc., Tewksbury, Mass.), stored at 2-8° C. and used within 1 month of preparation.

1× PBST (0.05% TWEEN®20 in 1× PBS) was prepared by combining and mixing 10 mL TWEEN®20 and 2 L 10× PBS. The solution was brought to a final volume of 20 L with deionized water. This reagent was stored at room temperature and used within 3 months of the date of preparation.

### Serum Handling

Non human primate (NHP) serum was prepared by combining and mixing equal amounts of normal Cynomolgus Macaque serum and buffer. Aliquots were stored at −20° C.

1:1000 Hyper-Immune Sera preparation

1 μL of Hyper-Immune Anti-EDI200 (Edimer Pharmaceuticals, Cambridge, Mass.) was added to 9994, of NHP serum. The solution was aliquoted and stored at −70° C. Procedure

### Samples and Solutions of the Assay

The negative control (NC) was undiluted NHP serum. The stock positive control was hyperimmune sera obtained from a Tabby mouse diluted 1:1000 using the undiluted NHP serum. The quality control (QC) low (QCL), QC mid (QCM), and QC high (QCH) samples were prepared at dilutions of 1:40,000; 1:20,000; and 1:10,000, respectively (see Table 12). The QC samples were prepared by serially diluting the stock positive control serum with the undiluted NHP serum. The QC samples were divided into 12 batches (batch 1, 2, 3, 4, 7, 8, 9, 11, 12, 14, 16 and 17).

TABLE 12
Quality control reagent preparation

| QC | Transfer | Diluent | Dilution |
|---|---|---|---|
| 1QC High | 7 μL of 1:1000 Hyper-Im- | 63 μL of NHP | 1:10,000 |
| | mune Sera in NHP serum | serum | |
| 2QC Mid | 30 μL of 1QC High | 30 μL of NHP | 1:20,000 |
| | | serum | |
| 3QC Low | 20 μL of 2QC Mid | 20 μL of NHP | 1:40,000 |
| | | serum | |

### Plate Preparation and Electrochemiluminescence Assay Procedures

Reagents were warmed to room temperature prior to use. A bridging ligand binding method was utilized for this assay. In accordance with this method, 150 μL of blocking buffer was added to each well of a standard streptavidin Sector Imager 2400 assay plate (Meso Scale Discovery, Gaithersburg, Md.). The plate was then sealed and incubated at room temperature on an orbital shaker (low speed) for 60-90 minutes.

A storage plate was prepared by adding 30 μL of each unknown sample, QC sample or NC to assigned wells of a 96-well dilution plate. 45 μL of Assay Diluent and 150 μL EDI200 Mastermix were subsequently added to each well of the dilution plate. The storage plate was then sealed and incubated at room temperature on an orbital shaker (low speed) for about 1 hour.

At the completion of incubation, the streptavidin assay plate was washed manually 3 times with 300 μL of 1× PBST per well and the plate was tapped on absorbent material to remove residual liquid.

At the completion of incubation of the storage plate, 50 μL was transferred from Blank, QC and unknown sample wells to wells of the streptavidin assay plate (Blank and QC samples were transferred in triplicate, unknown samples were transferred in duplicate). The streptavidin assay plate was then sealed and incubated at room temperature on an orbital shaker at a low setting for about 120 minutes.

After incubation, the streptavidin assay plate was washed manually 3 times with 300 μL of 1× PBST per well and tapped on an absorbent material to remove residual liquid.

2× Read Buffer T (Tris-based buffer with tripropylamine as a co-reactant for light generation, Meso Scale Discovery, Gaithersburg, Md.) was prepared by diluting 4× Read Buffer T with an equal volume of deionized water. 150 μL of 2× Read Buffer T was added to each well of the streptavidin assay plate. The plate was then analyzed using a Sector Imager 2400 (Meso Scale Discovery, Gaithersburg, Md.) within 20 minutes.

In this assay, the streptavidin plate is saturated, then a mix of biotinylated EDI200+anti-EDI200-containing serum+SulfoTag-EDI200 is added. Biotinylated EDI200 is captured. The Sulfotag-EDI200 is only captured if an anti EDI200 antibody is present to cross-link the SulfoTag-EDI200 to immobilized biotinlyated EDI200 b. The SulfoTag can be detected by electrochemiluminescence.

### Results

### Matrix Testing and NCO Determination

The 25 individual serum lots were analyzed in undiluted form. Samples were analyzed in triplicate over two runs on different days. Results from batch 1 were not used to calculate the negative cut off (NCO) because additional lots of serum were included in later batches. The NCO is defined as the response level at or below which the sample is considered negative. To normalize for run-to-run assay variation, a corrective factor (CF) was determined according to appropriate statistical procedures (Shankar, G. et al., Recommendations for the validation of immunoassays used for detection of host antibodies against biotechnology products. J Pharm Biomed Anal. 2008 Dec. 15; 48(5):1267-81. Epub 2008 Sep. 19). The CF for this assay was found to be 44.4. The NCO for each plate run for this method was determined by adding the CF to the mean negative control signal. Any response above this level was considered to be a positive response. The matrix testing results are presented below in Table 13.

TABLE 13
Matrix testing results

| | |
|---|---|
| Batch 2 | Batch 3 |

| | Mean | | Mean | |
|---|---|---|---|---|
| Serum Lot | Signal | % CV | Signal | % CV |
| 103518 | 115 | 5 | 130 | 0 |
| 103519 | 77 | 5 | 79 | 3 |
| 103520 | 119 | 10 | 124 | 2 |
| 103521 | 80 | 3 | 79 | 6 |
| 103522 | 130 | 4 | 128 | 2 |
| 103523 | 97 | 2 | 89 | 2 |
| 103524 | 108 | 4 | 101 | 1 |
| 103525 | 115 | 11 | 103 | 1 |
| 103526 | 96 | 9 | 96 | 2 |
| 103527 | 197 | 3 | 226 | 2 |
| 103528 | 100 | 6 | 94 | 5 |
| 103529 | 104 | 6 | 97 | 5 |
| 103530 | 134 | 2 | 150 | 2 |
| 103531 | 117 | 4 | 122 | 5 |
| 103532 | 97 | 5 | 95 | 2 |
| 103533 | 134 | 6 | 129 | 4 |
| 103534 | 116 | 6 | 119 | 7 |
| 103535 | 85 | 4 | 94 | 7 |
| 103536* | 1370 | 6 | 1516 | 6 |
| 103537 | 88 | 9 | 89 | 6 |
| 89585 | 89 | 5 | 87 | 5 |
| 89589 | 75 | 1 | 79 | 2 |
| 89591 | 93 | 3 | 96 | 4 |
| 89601 | 73 | 8 | 81 | 7 |
| 89602 | 99 | 6 | 94 | 6 |

*Results from this lot were excluded from NCO calculations, and this lot was excluded from subsequent serum pools.

### Control Sample Range Testing

Stability is assessed by comparing the raw signal acquired on the QC preparations following applicable storage conditions to validated ranges for signal to noise (S/N) ratios for the QC samples. Range testing for control samples was performed over three replicate batches (batches 5, 6, and 13), across 3 days, by two separate analysts. Each batch plate consisted of 32 wells of NC, QC low, and QC high. The minimum and maximum response value for each control sample was determined per batch. For each batch, the following values were determined.
Maximum and minimum values for NC, QC low, and QC high
Maximum QC value/minimum NC value=maximum S/N ratio for each QC
Minimum QC value/maximum NC value=minimum S/N ratio for each QC
   The lowest minimum QC values and ratios and highest maximum QC values and ratios across the three batches comprised the range for each QC level. Based on these results, the QC low value range is 1107 to 2693 (ratio range is 6.9 to 29.3) and the QC high value range is 13924 to 22308 (ratio range is 87.0 to 257.2). These ratio ranges were used to determine stability of the positive control QC samples during stability testing. All control levels and batches met acceptance criterion for precision. The range testing results are presented below in Table 14.

TABLE 14
Control sample range testing results

| | | Minimum | | Maximum | |
|---|---|---|---|---|---|
| Batch | % CV | Signal | S/N Ratio | Signal | S/N Ratio |

Negative Control

| | | | | | |
|---|---|---|---|---|---|
| 5 | 5 | 85 | NA | 103 | NA |
| 6 | 4 | 92 | NA | 107 | NA |
| 13 | 13 | 93 | NA | 160 | NA |

QC Low (1:40,000)

| | | | | | |
|---|---|---|---|---|---|
| 5 | 9 | 1590 | 15.4 | 2279 | 26.8 |
| 6 | 7 | 1925 | 18.0 | 2693 | 29.3 |
| 13 | 9 | 1107 | 6.9 | 1719 | 18.5 |

QC High (1:10,000)

| | | | | | |
|---|---|---|---|---|---|
| 5 | 9 | 15328 | 148.8 | 21865 | 257.2 |
| 6 | 8 | 15777 | 147.4 | 22308 | 242.5 |
| 13 | 6 | 13924 | 87.0 | 17879 | 192.2 |

### Precision

For core validation batches, two sets of QC low, QC mid and QC high samples were run in triplicate wells per level on the plate. For all other batches, one set of QC samples were run in triplicate wells on the plate. Precision of the method was analyzed over three batches (batches 4, 7 and 9) across 3 days, by two separate analysts for a total of 18 results per QC level. Intra-assay precision, reflective of the variation of the same preparation across replicates, was calculated for each QC level within each independent batch. Inter-assay precision, reflective of the variation of multiple preparations of the same QC levels, was calculated from the values obtained across the three core validation batches. The assay meets the criterion for intra- and interassay precision. The intra-assay and inter-assay precision results are presented below in Table 15.

TABLE 15
Precision results

| | QC Low (1:40,000) | | QC Mid (1:20,000) | | QC High (1:10,000) | |
|---|---|---|---|---|---|---|
| | Mean | % | Mean | % | Mean | % |
| Batch | Signal | CV | Signal | CV | Signal | CV |
| 4 | 2154 | 6 | 6289 | 8 | 21528 | 5 |
| 7 | 2402 | 8 | 6636 | 3 | 20647 | 10 |
| 9 | 1766 | 6 | 4788 | 10 | 14944 | 17 |

Inter-assay statistics

| | | | | | |
|---|---|---|---|---|---|
| 2107 | 14 | 5904 | 16 | 19040 | 19 |

### Drug Tolerance

Drug tolerance is a measure of the effect of the free test article on the detection of the positive control antibody. In general, the timing of EDI200 administration as well as the half life of the test article is taken into consideration to ensure that the timing of immunogenicity sample collection is such that the presence of circulating EDI200 is minimized. Nonetheless, if an animal administered EDI200 has an immunogenic response (i.e. produces an antibody to EDI200), resultant serum samples may contain both EDI200 and anti-EDI200 antibodies. Therefore the effect of the free EDI200 on the ability of the assay to detect the positive response was tested. The QC low and QC high levels were analyzed after being spiked with the eight EDI200 concentrations. The results demonstrate that the presence of EDI200 in the QC samples interferes with the detection of the anti-EDI200 antibodies in the hyperimmune serum. However, at the 1000 ng/mL concentration in the QC high sample and at the 500 ng/mL concentration in the QC low sample, the mean signal remained above the plate NCO suggesting that a positive antibody response would still be detected even though EDI200 is present, therefore this represents the drug tolerance level of the assay. The results are presented below in Table 16.

TABLE 16
Drug tolerance results

| Mean | Plate | | QC Low (1:40,000) | | QC High (1:10,000) | |
|---|---|---|---|---|---|---|
| NC | NCO | EDI200 | Mean | % | Mean | % |
| Signal | Signal | (ng/mL) | Signal | CV | Signal | CV |
| 85.7 | 130.1 | 1000 | 119 | 4 | 248 | 4 |
| | | 500 | 164 | 4 | 602 | 1 |
| | | 250 | 290 | 5 | 1895 | 1 |
| | | 125 | 469 | 6 | 4809 | 2 |
| | | 62.5 | 831 | 6 | 9549 | 2 |
| | | 31.25 | 1193 | 4 | 13558 | 3 |
| | | 15.63 | 1474 | 3 | 16914 | 2 |
| | | 7.81 | 1673 | 3 | 19334 | 2 |
| | | 0* | 2154 | 6 | 21528 | 5 |

*Mean and % CV of QC sets A and B from batch 4.

### Sensitivity

Serum from a Tabby mouse immunized with Fc-EDA1 further diluted into non-human primate serum was used to prepare the QC samples throughout the validation. The use of this hyperimmune serum demonstrates that the assay detects an antibody response against EDI200. Initially, a donkey anti-human IgG antibody was used as a surrogate positive control because it was expected that the immune response in immunized animals would also be against the human Fc portion of EDI200. However, during initial sensitivity and specificity testing, this surrogate antibody did not elicit a measurable response in NHP serum. The lack of response was likely due to the donkey anti-human IgG antibody binding to the non-human primate serum IgG, and not being available for binding to the Fc portion of EDI200. As an alternate method for measuring sensitivity and specificity of the method, a commercially available anti-EDA-1 antibody, Renzo-1, was utilized as a surrogate positive control.

Use of the surrogate positive control provides sensitivity in mass units. Renzo-1 was diluted using the undiluted serum. A total of seven dilutions for final concentrations ranging from 500 to 7.81 ng/mL were utilized and analyzed in triplicate. The sensitivity of this assay was considered to be 62.5 ng/mL, which resulted in a mean signal value above the plate NCO of 129.4. The sensitivity results using Renzo-1 are presented below in Table 17.

TABLE 17
Sensitivity results

| Mean NC | Plate NCO | Renzo-1 | Mean | |
|---|---|---|---|---|
| Signal | Value | (ng/mL) | Signal | % CV |
| 85.0 | 129.4 | 500 | 949 | 5 |
| | | 250 | 495 | 7 |
| | | 125 | 306 | 8 |
| | | 62.5 | 176 | 5 |
| | | 31.25 | 129 | 4 |
| | | 15.63 | 102 | 9 |
| | | 7.81 | 89 | 3 |

### Specificity

Specificity of the assay was tested to show that the observed positive response is EDI200 specific. This was achieved by comparing the surrogate positive control spiked in undiluted serum and used during sensitivity testing with a similarly prepared irrelevant monoclonal antibody (mouse anti-human CD106 IgG1 antibody), in the presence and absence of 1 μg/mL EDI200.

The reason for choosing to compare the surrogate positive control with the irrelevant antibodies is twofold. Firstly, the drug tolerance testing that utilized the anti-EDI200 hyperimmune serum indicated that the addition of EDI200 interferes with the ability of the assay to detect a positive response and therefore the positive signal of the surrogate control should also be reduced with the addition of EDI200. Secondly, the use of the surrogate control Renzo-1 antibody with mass units allowed for a direct comparison in antibody concentration with the irrelevant antibody (anti-CD106). The surrogate and irrelevant antibodies were tested at seven concentrations ranging from 500 to 7.81 ng/mL, the same concentration levels utilized during sensitivity testing. The presence of EDI200 in the surrogate control samples significantly decreased detection of the surrogate positive control as demonstrated by substantially lower mean signals in samples spiked with EDI200 compared to the same antibody concentration alone. The surrogate sample prepared at 500 ng/mL concentration tested positive in the presence of 1 μg/mL EDI200. Samples prepared using the irrelevant monoclonal antibody, tested negative with the mean signals below the NCO of 129.4 and within or below negative control ranges found during assay range determination whether tested in the presence or absence of EDI200. This demonstrates that the method is specific to anti-EDI200 antibodies and related surrogate antibodies. The specificity results using Renzo-1 are presented below in Table 18.

TABLE 18
Specificity results

| | | | | | |
|---|---|---|---|---|---|
| | Plate | | | | |
| Mean NC | NCO | Antibody | Mean | | Drug/Non- |
| Signal | Signal | (ng/mL) | Signal | % CV | drug Ratio* |

Renzo-1 antibody + 1 μg/mL EDI200

| | | | | | |
|---|---|---|---|---|---|
| 85.0 | 129.4 | 500 | 171 | 3 | 0.18 |
| | | 250 | 89 | 2 | 0.18 |
| | | 125 | 85 | 3 | 0.28 |
| | | 62.5 | 75 | 2 | 0.43 |
| | | 31.25 | 80 | 9 | 0.62 |
| | | 15.63 | 78 | 10 | 0.77 |
| | | 7.81 | 81 | 5 | 0.91 |

Irrelevant CD106 antibody alone

| | | | | | |
|---|---|---|---|---|---|
| 85.0 | 129.4 | 500 | 86 | 3 | NA |
| | | 250 | 83 | 3 | NA |
| | | 125 | 82 | 6 | NA |
| | | 62.5 | 81 | 1 | NA |
| | | 31.25 | 81 | 5 | NA |
| | | 15.63 | 81 | 5 | NA |
| | | 7.81 | 85 | 4 | NA |

Irrelevant CD106 antibody + 1 μg/mL EDI200

| | | | |
|---|---|---|---|
| 500 | 81 | 4 | 0.95 |
| 250 | 80 | 6 | 0.96 |
| 125 | 76 | 2 | 0.93 |
| 62.5 | 76 | 4 | 0.94 |
| 31.25 | 76 | 1 | 0.93 |
| 15.63 | 74 | 4 | 0.91 |
| 7.81 | 80 | 3 | 0.93 |

*Calculated using results from sensitivity testing table (Table 17).

### Stability

Stability was assessed by comparing the raw signal acquired on the QC preparations following applicable storage conditions to the acceptable ranges for the QC low/negative control ratio and QC high/negative control ratio. In addition, each QC set must meet acceptance criteria for precision.

### Short Term Stability

A set of aliquots were prepared from each of the two QC low and QC high concentrations from batch 7. Short term stability testing was assessed by storing the two sets of QC samples at ambient conditions for at least 4 hours prior to analysis. The short term stability results are presented below in Table 19.

TABLE 19
Short term stability results

| | | QC Low (1:40,000) | | | QC High (1:10,000) | | |
|---|---|---|---|---|---|---|---|
| Storage | | Mean | % | S/N | Mean | % | S/N |
| Condition | Replicate | Signal | CV | Ratio | Signal | CV | Ratio |
| Ambient | A | 1955 | 5 | 22.1 | 19898 | 2 | 225.3 |
| | B | 1738 | 5 | 19.7 | 17461 | 2 | 197.7 |

All short term stability samples met acceptance criterion for precision. Mean signal values and/or ratios for the stability QC samples were within the validated ranges, therefore, samples are considered stable for at least 4 hours at ambient conditions.

### Freeze/Thaw Cycle Stability

Two sets of aliquots were prepared from each of the two QC low and QC high concentrations from batch 4. One set was stored frozen at −10 to −30° C., and the second set was stored frozen at −50 to −90° C. Freeze/thaw stability testing was performed by thawing and re-freezing QC samples up to three times. Following the third freeze/thaw cycle, the samples were analyzed. The freeze/thaw cycle stability results are presented below in Table 20.

TABLE 20
Freeze/thaw cycle stability results

| | | QC Low (1:40,000) | | | QC High (1:10,000) | | |
|---|---|---|---|---|---|---|---|
| Frozen Cycle | | Mean | % | S/N | Mean | % | S/N |
| Condition | Replicate | Signal | CV | Ratio | Signal | CV | Ratio |
| −10 to −30° C. | A | 1982 | 5 | 22.4 | 18927 | 3 | 214.3 |
| | B | 1964 | 4 | 22.2 | 18586 | 1 | 210.4 |
| −50 to −90° C. | A | 1994 | 2 | 22.6 | 18129 | 2 | 205.2 |
| | B | 1886 | 1 | 21.4 | 18143 | 2 | 205.4 |

All freeze/thaw cycle stability samples met acceptance criterion for precision. Mean signal values and/or ratios for the stability QC samples were within the validated ranges, therefore, samples are considered stable for up to three freeze/thaw cycles at both storage temperatures.

### Long Term Storage Stability

Two sets of aliquots were prepared from each of the two QC low and QC high concentrations from batch 4. One set was stored frozen at −10 to −30° C., and the second set was stored frozen at −50 to −90° C. After the required frozen storage intervals, the samples were thawed and analyzed. The long term storage stability results are presented below in Table 21.

TABLE 21
Long term storage stability results

| | | QC Low (1:40,000) | | | QC High (1:10,000) | | |
|---|---|---|---|---|---|---|---|
| Storage | | Mean | % | S/N | Mean | % | S/N |
| Condition | Replicate | Signal | CV | Ratio | Signal | CV | Ratio |

7 day

| | | | | | | | |
|---|---|---|---|---|---|---|---|
| −10 to −30° C. | A | 2697 | 2 | 30.2 | 18382 | 2 | 205.8 |
| | B | 2151 | 3 | 24.1 | 18319 | 1 | 205.1 |
| −50 to −90° C. | A | 2064 | 6 | 23.1 | 16563 | 9 | 185.4 |
| | B | 2041 | 3 | 22.8 | 16982 | 5 | 190.1 |

28 day

| | | | | | | | |
|---|---|---|---|---|---|---|---|
| −10 to −30° C. | A | 2232 | 1 | 24.1 | 21298 | 2 | 229.8 |
| | B | 2168 | 3 | 23.4 | 21315 | 1 | 230 |
| −50 to −90° C. | A | 1864 | 2 | 20.1 | 18106 | 2 | 195.4 |
| | B | 1994 | 2 | 21.5 | 20837 | 2 | 224.9 |

42 day

| | | | | | | | |
|---|---|---|---|---|---|---|---|
| −10 to −30° C. | A | 1717 | 6 | 20.3 | 16355 | 9 | 193.2 |
| | B | 1721 | 8 | 20.3 | 16125 | 6 | 190.4 |
| −50 to −90° C. | A | 1731 | 8 | 20.4 | 14498 | 7 | 171.2 |
| | B | 1620 | 10 | 19.1 | 14596 | 2 | 172.4 |

All long term storage stability samples met acceptance criterion for precision. Mean signal values and/or ratios for the stability QC samples stored at −50 to −90° C. were within the validated ranges throughout long term stability testing throughout long term stability testing. The mean signal value and ratio for the 7 day QC low replicate A stored at −10 to −30° C. was slightly above validated range. This was considered normal variability and did not indicate a lack of stability. Therefore, samples are considered stable for up to 42 days at both temperatures.

### Conclusion

A semi-quantitative immunogenicity method was successfully validated to detect the presence of anti-EDI200 antibodies in non-human primate serum. The assay correction factor was determined during matrix testing using 24 lots of non-human primate serum procured from a commercial source. The negative cut off (NCO) for the method was estimated such that 95% of all the other observations fall below it (i.e. 95% upper limit). The correction factor was calculated using the formula Correction Factor=NCO−mean response of individual sera. The correction factor was therefore determined to be 44.4 and was added to each individual plate's mean signal of negative control values to determine the cut-off for each individual run. The assay is specific to anti-EDI200 antibodies. The QC low positive control samples can be detected in the presence of 500 ng/mL EDI200, and QC high positive control samples can be detected in the presence of 1000 ng/mL EDI200. During sensitivity testing, the surrogate control antibody yielded a response above the NCO at a concentration of 62.5 ng/mL.

Therefore the assay sensitivity limit is 62.5 ng/mL and higher. The assay performed well within its acceptance criteria for precision and reproducibility.

### Example 4

### EDI200 Vehicle: Intravenous Study in Non Human Primates

The objective of this study was to evaluate the tolerability of the vehicle for EDI200 when administered as an intravenous infusion in adult monkeys.

TABLE 22
Study design

| | Dose | Dose | Dose | | | |
|---|---|---|---|---|---|---|
| | Level | Volume | Volume | Dose | Number of | |
| | (mg/kg/ | (mL/kg/ | (mL/kg/ | Conc. | Animals | |
| Group | dose) | dose) | hr) | (mg/mL) | M | F |
| 1 | 0 | 20 | 10 | 0 | 1 | 1 |

Each dose was administered via intravenous infusion

### Materials and Methods

Sterile vehicle for EDI200 was thawed overnight and administered. The vehicle contained 20 mM sodium phosphate, 300 mM NaCl, pH 7.2, and 0.02% TWEEN®20 (Sigma-Aldrich, St. Louis, Mo.) (w/v). An intravenous syringe pump system was used to administer the vehicle to the animals in a peripheral vein, while restrained in a recumbent sling.

Two non-naive Cynomolgus monkeys (one male and one female) were transferred from the training colony at MPI Research and acclimated to the dosing procedures prior to study initiation. A staff veterinarian was present during the dosing period. Detailed clinical evaluations were performed at transfer, approximately 30 minutes following the start of infusion and at approximately 3 hours after the end of infusion. Body weights were recorded at transfer and prior to dose administration. Vital signs, including blood pressure, heart rate, respiration rate and body temperature were recorded prior to the start of infusion, at approximately 30 minutes after start of infusion and approximately 3 hours after the end of infusion. The animals were returned to the colony after completion of all study related procedures.

### Results

Both animals survived the single dose of vehicle administered. There were no adverse clinical findings observed during or after the dosing period.

The male (animal number 1001) weighed 3.37 kg and the female (animal number 1501) weighed 3.64 kg on Day −1 (prior to dosing). These weights were used to calculate the dose and corresponding intravenous infusion rate.

There were no adverse changes in the vital signs collected during or after intravenous administration of the vehicle to indicate the development of an allergic or anaphylactic reaction. The blood pressure values did not produce any consistent changes that could be correlated with a significant hypotensive reaction. The vital signs recorded are listed in Table 23.

TABLE 23
Vital signs

| | | Male | Female |
|---|---|---|---|
| Description | | (Animal # 1001) | (Animal # 1501) |
| Heart Rate | Time 1 | 236 | 260 |
| (beats/min) | Time 2 | 211 | 246 |
| | Time 3 | 219 | 243 |
| Respiration Rate | Time 1 | 76 | 44 |
| (breaths/min) | Time 2 | 60 | 52 |
| | Time 3 | 68 | 52 |
| Temperature (° C.) | Time 1 | 38.4 | 38.8 |
| | Time 2 | 37.0 | 37.8 |
| | Time 3 | 37.9 | 38.2 |
| Blood Pressure | Time 1 | 145/77 | 150/88 |
| (mm Hg) | Time 2 | 134/84 | 135/73 |
| | Time 3 | 132/76 | 136/92 |
| Mean Arterial | Time 1 | 108 | 120 |
| Pressure | Time 2 | 97 | 99 |
| | Time 3 | 109 | 103 |

Time 1 = Predose
Time 2 = 30 minutes after start of infusion
Time 3 = 3 hours following end of infusion

### Conclusion

The vehicle for EDI200, when administered once by intravenous infusion at a dose volume of 10 mL/kg/hr to non-naive monkeys, did not produce any adverse clinical findings or any changes in the vital signs that would indicate the possibility of an allergic or anaphylactic reaction. The results of this study confirmed that the vehicle was well tolerated by the adult monkeys under the conditions tested.

### Example 5

### Intravenous Infusion Study in Non-Human Primates

A study was conducted to evaluate EDI200 following twice weekly intravenous (IV) infusion doses, in the context of toxicity, reversibility, progression, or delayed appearance of any observed changes following a 15-day postdose observation period.

One treatment group of three male and three female Cynomolgus monkeys and one treatment group of five male and five female monkeys were administered the test article at respective dose levels of 30 or 100 mg/kg/dose twice weekly for 3 weeks. One additional group of five animals/sex served as the control group and received the vehicle (20 mM sodium phosphate, 300 mM NaCl, pH 7.2, and 0.02% TWEEN®20 (Sigma-Aldrich, St. Louis, Mo.) (w/v)). The dose volume for all groups was 20 mL/kg/dose (10 mL/kg/hr). Following the treatment period, two animals at 0 and 100 mg/kg/dose were maintained for a 15-day recovery period.

Observations for morbidity, mortality, injury, and the availability of food and water were conducted twice daily for all animals. Clinical observations were conducted twice weekly during the treatment period and weekly during recovery. Body weights were measured and recorded weekly. Ophthalmoscopic examinations were conducted pretest and prior to each scheduled necropsy. Electrocardiographic examinations were conducted pretest, on Day 15, and prior to the recovery necropsy. Blood samples for determination of the serum concentrations of the test article were collected from all animals at designated time points on Days 1 and 19, and prior to the recovery necropsy. The toxicokinetic (TK) parameters were determined for the test article from concentration-time data in the test species. Blood samples for immunogenicity evaluations by enzyme-linked immunosorbent assay (ELISA) and blood and urine samples for clinical pathology evaluations were collected pretest and prior to the terminal and recovery necropsies. At the terminal and recovery necropsies, examinations were performed, organ weights were recorded, and tissues were microscopically examined. Gross lesions only were examined microscopically at the recovery necropsy.

The low-dose formulation (30 mg/kg/dose) was found to be prepared at the targeted concentration, based on the results of the analytical evaluation. The high-dose formulation (100 mg/kg/dose) was not analyzed, but used as received without dilution. No test article was found in the controls samples analyzed.

### Materials and Methods

### Vehicle and Test Article Preparation

Fresh vehicle (20 mM sodium phosphate, 300 mM NaCl, pH 7.2, and 0.02% TWEEN®20 (w/v)) was prepared. Vehicle formulations were prepared weekly for study use under a laminar flow hood using aseptic technique, dispensed for use on the day of administration, and were stored refrigerated at 2 to 8° C. when not in use.

The bulk test article, EDI200, was used as received from Althea Technologies (San Diego, Calif.) and stored frozen at −50 to −90° C. No adjustment was made for purity when preparing the test article formulations. The test article was administered undiluted or was diluted with sterile vehicle to achieve the desired dose volumes. The frozen stock solution was thawed under a laminar flow hood using aseptic technique and went through a maximum of two freeze-thaw cycles. An appropriate amount of the stock solution was thawed for use or dilution into designated volumes fresh for each preparation. Formulations of the test article were prepared fresh for each concentration on the day of administration at nominal concentrations of 1.5 and 5 mg/mL, and were stored refrigerated at 2 to 8° C. when not in use.

### Animal Acquisition and Acclimation

A total of 15 male and 15 female experimentally naive Cynomolgus monkeys, approximately 2 years 5 months to 4 years 1 month of age at transfer, were transferred from an MPI Research, Inc. stock colony. During acclimation as part of the stock colony, the monkeys were examined by a clinical veterinarian, weighed, and observed daily with respect to general health and any signs of disease. Clinical pathology, stool flotation, and intrapalpebral tuberculin tests were performed, and the animals were considered suitable prior to being released from quarantine.

During the 14-day acclimation period, the animals were observed daily with respect to general health and any signs of disease. All animals were given a detailed physical examination, detailed clinical examination, and body weights and body temperatures were measured prior to selection for study. The animals were acclimated to the sling apparatus on three occasions prior to test article administration.

### Randomization, Assignment to Study, and Maintenance

Using a standard, by weight, measured value randomization procedure, 13 male and 13 female animals (weighing 2.32 to 2.85 kg and 2.92 to 3.45 kg, respectively, at randomization) were assigned to the control and treatment groups identified in Table 24.

TABLE 24
Group assignments

| | | Number of | | | |
|---|---|---|---|---|---|
| Group | Dose Level | Animals | | Animal Numbers | |
| Number | (mg/kg/dose) | Male | Female | Male | Female |
| 1 | 0 | 5 | 5 | 1001-1005 | 1501-1505 |
| 2 | 30 | 3 | 3 | 2001, 2002, | 2501-2503 |
| | | | | 2003 | |
| 3 | 100 | 5 | 5 | 3001-3005 | 3501-3505 |

*Following the treatment period, two animals were maintained for a 15-day recovery period.

Animals assigned to study had body weights within ±20% of the mean body weight for each sex. Extra animals obtained for the study, but not placed on study, were returned to the stock colony.

On Day 1, prior to dosing, one male at 30 mg/kg/dose (animal number 2001) was replaced due to excessive body weight. A single monkey was utilized as a replacement animal and was assigned a unique animal number (2101). All data for the replaced animal are not reported but are maintained in the study data.

Upon receipt, during quarantine, and during testing monkeys were social-housed in groups of two, three, or four (single-sex) in stainless steel appropriately-sized cages in an environmentally controlled room. Monkeys were individually housed for required individual data collection. The monkeys were provided environmental enrichment during the quarantine and study, as documented in the data.

Fluorescent lighting was provided for approximately 12 hours per day. The dark cycle was interrupted intermittently due to study-related activities. Temperature and humidity were continuously monitored, recorded, and maintained to the maximum extent possible within the protocol-designated ranges of 64 to 84° F. and 30 to 70%, respectively. The actual temperature and humidity findings are not reported but are maintained in the study file.

Lab Diet (Certified Primate Diet, PMI Nutrition International, Inc., St. Louis, Mo.) was available to the monkeys twice a day, except during designated periods. PrimaTreats (Bio Serv, Frenchtown, N.J.) were offered twice a day and other enrichment foods were provided on a regular basis. These offerings were documented in the study records. The lot number from each diet lot used for this study was recorded. Certification analysis of each diet lot was performed by the manufacturer. Tap water was available ad libitum via an automatic watering system. On occasion, animals were offered supplemental food (e.g., fruity gems, marshmallow fluff, peanut butter, apples, grapes, and sweet potatoes) per veterinarian recommendation. The water supply was monitored for specified contaminants at periodic intervals according to standard operating procedures.

### Test Article Administration

The vehicle and test article were administered twice weekly for 3 weeks (Days 1, 5, 8, 12, 15, and 19) as an intravenous infusion at 10 mL/kg/hour for a maximum dose of 20 mL/kg via a percutaneous peripheral vein catheter. The dose levels were 0, 30, and 100 mg/kg/dose.

The animals were restrained in a sling apparatus for dosing. Prior to catheter placement, the area over the suitable vein was shaved as needed and cleansed with chlorhexidine scrub. Doses were administered using an infusion pump and sterile disposable reservoirs.

Individual doses were withdrawn into appropriately labeled reservoirs. The dosing reservoirs were filled with the appropriate volume (dose volume+extra) required for dosing on that day. The actual volume infused was calculated and adjusted based on the most recent body weight of each animal. Dose accountability was performed by weighing the reservoir prior to the start and at the end of each infusion.

### Results

### Analysis of Dosing Formulations Concentration

The results from formulation analysis (Table 25) showed the values to be within the expected acceptance criteria for this method (±10% of nominal). The average recovery rates at 30 mg/kg/dose ranged from 95.0 to 97.5%. This confirmed that the formulations were properly prepared and the animals received the appropriate dose levels. The aliquots from the control group were below the level of quantitation and confirmed to be devoid of test article. The concentration from the high-dose group (100 mg/kg/dose) was not analyzed, as it was used as received without dilution.

TABLE 25
Formulation analysis

| | Nominal | Average | | |
|---|---|---|---|---|
| | Concen- | Calculated | | % Relative |
| Dose Level | tration | Conc.^{a} | Average % | Standard |
| (mg/kg/dose) | (mg/mL) | (mg/mL) | Recovery^{a,b} | Deviation^{a} |
| 0 | 0.00 | BLQ | NA | NA |
| 30 | 1.50 | 1.4243-1.4623 | 95.0-97.5 | 0.250-2.361 |

^{a}Results are the range of values determined during Weeks 1-3.
^{b}Average % recovery was calculated from the nominal concentration.
NA—Not Applicable
BLQ—Below the Limit of Quantitation (<0.05 Absorbance Units)

### Mortality

All animals survived to scheduled necropsy on Day 22 for the terminal animals and on Day 37 for the recovery animals.

### Clinical Observations

No test article-related clinical observations were observed during the course of the study. A finding of sparse hair was documented in all dose groups; however, these findings were generally present prior to the dosing phase. At 100 mg/kg/dose soft feces was seen in three males on Day 3 and three females on Day 16. As this finding was limited to a single timepoint for each sex and there was no consistent pattern in the timing, this finding was not considered test article related. No test article-related changes in body weight, ophthalmoscopic findings or treatment-related effects on the ECG parameters were documented during the study period.

### Clinical Pathology

No test article-related effects were observed on hematology parameters, coagulation parameters, clinical chemistry analytes or urinalysis parameters at termination or recovery.

### Serum Analysis

A plate-based ligand binding method (ELISA) was used for the detection of EDI200 in serum. The incurred sample reanalysis (ISR) evaluation demonstrated that more than two-thirds of the total samples tested were within 30% of their original value. Therefore, the ISR results were considered to be acceptable.

### Toxicokinetic Analysis

There were no measurable serum concentrations of EDI200 in control animals on Day 1 or 19, or at the end of a 2-week recovery period. The toxicokinetic parameters for EDI200 were similar in male and female monkeys on Day 1 and Day 19.

Measurable concentrations of EDI200 were present in monkeys treated with EDI200 at 30 and 100 mg/kg/dose after the first dose on Day 1 and the last dose on Day 19. Prior to the last day of dosing on Day 19, EDI200 was found in serum at low levels in both male and female animals at 30 and 100 mg/kg/dose. Measurable concentrations of EDI200 were found at the end of the 2-week recovery period at 100 mg/kg/dose in 1 of 2 males and 1 of 2 females.

Systemic exposure to EDI200, as estimated by AUC_{0-∞}, AUC₀₋₇₂, and AUC₀₋ₜₗₐₛₜ, increased more than in proportion to dose between 30 and 100 mg/kg/dose. Clearance (CL) and (volume of distribution at steady state) V_{SS} were lower after 100 mg/kg/dose than after 30 mg/kg/dose.

Half-life was longer after 100 mg/kg/dose than after 30 mg/kg/dose on Day 1, but t_{1/2} was similar at both dose levels on Day 19 (see Table 26).

TABLE 26
Toxicokinetic parameters for EDI200

| | Dose | | | | | |
|---|---|---|---|---|---|---|
| | (mg/kg/ | | AUC₀₋ₗₐₛₜ | CL | t_{1/2} | Vₛₛ |
| Day | dose) | Sex | (hr*ng/ml) | (mL/min/kg) | (hr) | (mL/kg) |
| 1 | 30 | M | 1070000 ± 149000 | 0.467 ± 0.0590 | 11.5 ± 0.663 | 322 ± 727 |
| | | F | 1310000 ± 419000 | 0.404 ± 0.127 | 10.5 ± 0.672 | 293 ± 110 |
| | | Combined | 1190000 ± 312000 | 0.436 ± 0.0951 | 11.0 ± 0.792 | 308 ± 848 |
| | 100 | M | 8740000 ± 2130000 | 0.189 ± 0.0490 | 48.4 ± 5.89 | 172 ± 23.5 |
| | | F | 8600000 ± 3080000 | 0.206 ± 0.0955 | 44.7 ± 13.3 | 220 ± 89.7 |
| | | Combined | 8670000 ± 2500000 | 0.197 ± 0.0721 | 46.8 ± 9.36 | 196 ± 66.7 |
| 19 | 30 | M | 753000 ± 589000 | 0.950 ± 0.651 | 14.7 ± 16.9 | 365 ± 201 |
| | | F | 1980000 ± 310000 | 0.228 ± 0.0373 | 24.1 ± 7.03 | 417 ± 113 |
| | | Combined | 1370000 ± 792000 | 0.589 ± 0.571 | 19.4 ± 12.7 | 391 ± 149 |
| | 100 | M | 7470000 ± 798000 | 0.223 ± 0.0236 | 17.3 ± 8.06 | 99.1 ± 24.9 |
| | | F | 6410000 ± 2820000 | 0.305 ± 0.149 | 17.0 ± 16.1 | 125 ± 57.5 |
| | | Combined | 6940000 ± 2030000 | 0.264 ± 0.109 | 17.2 ± 12.0 | 112 ± 43.9 |

### Immunogenicity Analysis

The qualitative immunogenicity assay (ELISA) utilized a plate-based negative cut off (NCO) to determine whether study samples contained anti-EDI200 antibodies (Shankar, G. et al., J Pharm Biomed Anal. 2008 Dec. 15; 48(5):1267-81. Epub 2008 Sep. 19). The plate-based NCO was calculated using the sum of the mean luminescence signal for the negative control (undiluted serum) samples on the plate and an assay specific corrective factor (CF). The assay specific CF was determined during validation and was found to be 44.4. The CF was calculated by subtracting the arithmetic mean of the serum data from a robust cutpoint determined through estimation of the 95^{th} percentile for the luminescence signals from 24 individual serum lots tested over two batches. This method allowed for an approximate 5% false positive response rate. The relative strength of a positive antibody response was assessed by dividing the mean response for the sample by the negative control response or signal:noise (S/N) ratio.

The pretest serum samples for all monkeys except one female were ELISA negative for anti-EDI200 antibodies. The pretest serum sample for this animal was slightly ELISA positive with a S/N ratio of 1.92. This result is not considered meaningful due to a lack of a “normal range” in pre-dosing unexposed animals. This sample was not reanalyzed to confirm the original result. This slight ELISA positive result was likely a false positive response.

Two of five males at 0 mg/kg/dose were ELISA positive for anti-EDI200 antibodies at the terminal collection. One of three males at 30 mg/kg/dose was ELISA positive for anti-EDI200 antibodies at terminal collection. Two of five males and three of five females at 100 mg/kg/dose were ELISA positive for anti-EDI200 antibodies at the terminal collection. All animals that were ELISA positive at the terminal collection were part of the terminal necropsy, so persistence or recovery could not be assessed in those individual animals. One of two females at 0 mg/kg/dose was ELISA positive for anti-EDI200 antibodies at the recovery collection. One of two males and two of two females at 100 mg/kg/dose were ELISA positive for anti-EDI200 antibodies at the recovery collection. The positive samples from the terminal and recovery collections were reanalyzed and the reanalysis results confirmed the original ELISA positive results for all samples. The luminescence signals in the reanalysis batch (batch 3) were consistently higher than the original results (batch 2) most likely due to plate-to-plate variability and the batches being analyzed 18 days apart. Therefore, additional interpretation will be made only using the original results.

The 0 mg/kg/dose males had only slight ELISA positive responses with S/N ratios of ≦2.0. The 0 mg/kg/dose female had a stronger response (S/N ratio=6.5).

The bioanalytical results showed that the 0 mg/kg/dose animals were not exposed to EDI200 at any point during the study, as demonstrated by BLQ results at 1 hour postdose on Days 1 and 19, as well as at the end of the recovery interval. Therefore, the positive responses seen in these animals are likely due to non-specific binding, especially in the female animal.

The 30 mg/kg/dose male had a S/N ratio of 10.5. The terminal 100 mg/kg/dose males had S/N ratios of 2.9 and 4.4, while the recovery male had a S/N ratio of 15.3. The terminal 100 mg/kg/dose females had S/N ratios of 1.6, 4.1, and 7.0, while the recovery females had S/N ratios of 2.8 and 3.9. Given the large variability in S/N ratio values and the results from the 0 mg/kg/dose animals, it cannot be determined conclusively if the positive responses seen in treated animals was due to non-specific binding, a true anti-EDI200 antibody response, or a combination of both. Most of the anti-EDI200 negative animals in the EDI200 treated groups had EDI200 levels >2000 ng/mL at 72 hours postdose. Treated animals with positive anti-EDI200 responses had low level or no systemic EDI200 at 72 hours postdose. Therefore, it is possible that circulating EDI200 masked or depleted anti-EDI200 responses.

### Postmortem Study Evaluations

There were no test article-related macroscopic findings or organ weight changes. At the terminal necropsy, pituitary gland weights (absolute and relative to body and brain weights) were slightly higher than controls (with or without statistical significance) in males at 30 and 100 mg/kg/dose. On the other hand, pituitary gland weights relative to body weights were lower in females at 30 and 100 mg/kg/dose compared to controls. Only the value at 30 mg/kg/day was statistically identified. These changes were considered the result of normal biological variation based on the lack of dose response.

At the terminal necropsy, epididymides and testes weights (absolute and relative to body and brain weights) were higher in males at 30 and 100 mg/kg/dose compared to controls. The change was related to various degrees of sexual immaturity and was not considered to be test article related.

At the terminal necropsy, ovary weights (absolute and relative to body and brain weights) were lower in females at 30 and 100 mg/kg/dose compared to controls. The change was related to variation in the estrus cycle and was not considered to be test article related.

At the terminal necropsy, thymus weights (absolute and relative to body and brain weights) were lower in females at 30 and 100 mg/kg/dose compared to controls. The change was considered the result of normal biological variation based on the lack of microscopic correlates.

At the recovery necropsy, the following organ weights (absolute and/or relative to body and brain) at 100 mg/kg/day were different from controls: adrenal glands, pituitary gland, and mandibular salivary gland in males, and thymus in females. These changes were related to normal biological variation based on the small group size.

### Conclusion

Intravenous infusion of EDI200 at 30 and 100 mg/kg/dose to nonhuman primates twice a week for three weeks did not produce any test article-related clinical observations, changes in body weight, ophthalmoscopic findings, changes in the ECG parameters, clinical pathology results, organ weights, or macroscopic necropsy findings. The only test article-related microscopic observations included findings of minimal to mild epidermal hyperplasia, minimal to mild subacute/chronic inflammation, and minimal to mild mononuclear cell infiltration at the infusion sites. These changes were not considered adverse based on their occurrence in some controls and the minimal to mild severity. The TK evaluation confirmed systemic exposure in the animals in a dose dependent manner.

Given the large variability in the signal to noise ratio values for the immunogenicity evaluation (anti-EDI200 antibodies) and the results from the control animals, it cannot be determined conclusively if the positive responses seen in treated animals were due to non-specific binding, a true anti-EDI200 antibody response, or a combination of both.

Based on the results obtained from this toxicity study in monkeys following twice weekly exposure to EDI200 at dose levels of 30 and 100 mg/kg/dose for three weeks, a No-Observed-Adverse-Effect-Level (NOAEL) for general toxicity was considered to be 100 mg/kg/dose, the highest dose level tested, based on the lack of any significant toxicologically relevant findings.

### Example 6

### Analysis of Effective EDI200 Treatment Through Gene Expression Analysis

Effective EDI200 treatment leads to activation of EDA receptor signaling and upregulation of EDA-A1 responsive genes. To determine the profile of such gene expression changes, skin biopsies taken from subjects undergoing EDI200 treatment are analyzed by quantitative PCR (qPCR) analysis for genome-wide changes in mRNA expression level in response to treatment.

### Mouse Studies

Initial studies were carried out in mice to look for changes in expression of EDA-A1-responsive genes in response to EDI200 treatment. After EDI200 injection treatment in neonates, expression of EDA receptor and sonic hedgehog (Shh) mRNA levels were increased (as compared to vehicle) before returning to baseline expression levels (Table 27).

TABLE 27
Ratio of mRNA Expression Compared to Vehicle (log base 2)

| | Back | Tail | Footpad |
|---|---|---|---|
| EDAR | 1.88 | 0.94 | 1.73 |
| Shh | 2.09 | 1.95 | 4.87 |
| Ptch1 | 2.06 | 2.24 | 1.92 |
| Ptch2 | 1.91 | 1.73 | 1.65 |
| Gli1 | 1.17 | 2.28 | 0.48 |

### Human Studies

In humans adult male XLHED subjects undergoing EDI200 treatment, gene expression analysis is performed to determine drug efficacy. Skin biopsies are collected from subject forearms prior to initial treatment, after the final treatment (five treatments in all) and after a 4 week recovery period. These samples undergo genome-wide analysis of mRNA expression such as with RNA-Seq technology and gene expression patterns are analyzed to detect EDA receptor activity. In some cases, entire genome patterns may be evaluated. In other cases, gene expression pathways associated with or believed to be associated with EDA or EDAR signaling pathways are evaluated.

### Example 7

### EDA Gene Analysis for XLHED

XLHED is inherited in an X-linked manner and caused by mutations in the EDA gene.

Most mutations are null mutations; however, some partial function missense mutations leading to milder dental phenotypes have been reported (Mikkola et al. 2008).

Sequence analysis of the EDA gene can identify mutations in the coding sequence and +15 bp and −15 bp into the intron sequence of each of the coding exons.

The test involves taking a tissue specimen; usually blood, to obtain a sample of DNA. Tissue samples and or the specimen may also include amniotic fluid. The determination of selection of tissue specimen may occur post-amniocentesis analysis of the mother (i.e., in utero). Testing may also be performed in family members of individuals known to, or suspected of, being affected by an ectodermal dysplasia such as XLHED.

This DNA is then used to determine the gene sequence of each of the associated genes. The method involves direct sequencing of the 8 coding exons of the EDA gene in a 384 well plate format. The patient's genetic sequence is then compared to the normal sequence to identify mutations that may be responsible for the clinical presentation of the patient. Comprehensive molecular testing involves sequencing as well as Multiplex Ligation-dependent Probe Amplification (MLPA) copy number analysis of the EDA gene.

Sequence analysis is used with the present invention to identify subjects who may benefit from treatment with compounds of the present invention. Such analysis is conducted when an individual is suspected of suffering from XLHED as evidenced by phenotypic characteristics such as hypotrichosis (sparse hair), hypohidrosis (reduced sweating) and hypodontia (absence of teeth). The hair is often thin, slow-growing, lightly pigmented scalp hair and sparse or missing eyebrows. Sweating is greatly deficient, which can lead to hypothermic episodes without environmental modifications used to control body temperature. Often there are only a few abnormally formed teeth that erupt at a late age. The teeth are typically smaller than average with conical crowns. Female carriers show mosaic patterns of sweat poor function and distribution, often some degree of hypodontia and some have mild hypotrichosis (Cambiaghi et al. 2000). Affected individuals may have other features including fragile appearing skin, raspy voice, decreased sebaceous secretions, abnormal nasal secretions and facial features such as frontal bossing, protruding lips, saddle nose and sunken cheeks. Sequence analysis is also used to determine the genotype of prenatal subjects carried by an individual suspected to carry genetic defects linked to XLHED.

### Example 8

### Development and Validation of a Ligand Binding Method to Detect EDI200 or Anti-EDI200 Antibodies in Human Serum

Development of a quantitative pharmacokinetic method to measure EDI200 or anti-EDI200 antibodies in human serum may be carried out using known methods in the art. Once developed, such an assay may undergo feasibility testing and/or further development. The method is then validated for use with human serum in a similar manner as the non-human primate methods taught herein. Method development and validation may be based, in part, on the Guidance for Industry: Bioanalytical Method Validation available through the FDA and conducted in accordance with the United States Food and Drug Administration (FDA) Good Laboratory Practice (GLP) Regulations, 21 CFR Part 58.

### Example 9

### Development and Validation of a Cell-Based Assay for the Detection of the anti-EDI200 Neutralizing Antibodies in Human Serum

Development of a cell-based assay for the detection of anti-EDI200 neutralizing antibodies in human serum may be carried out as described herein. Once developed, such an assay may undergo feasibility testing and/or further development. The method is then validated for use with human serum in a similar manner as the non-human primate methods taught herein. Method development and validation is based, in part, on the Guidance for Industry: Bioanalytical Method Validation available through the FDA and conducted in accordance with the United States Food and Drug Administration (FDA) Good Laboratory Practice (GLP) Regulations, 21 CFR Part 58. Further details on the development of such an assay are described here.

### Description of Matrix

Normal human serum is procured from a commercial source. A minimum of 20 lots of human serum are evaluated in matrix testing with the intention of using a pooled human serum source as blank matrix. Following validation the assay is bridged using 10-15 samples of serum from XLHED patients. A minimum of 10 lots of human serum are evaluated during bridging matrix testing with the intention of using a pooled human serum source as blank matrix during sample analysis.

### Neutralizing Antibody Assay Design

A cell-based neutralizing antibody assay is designed to measure the ability of anti-EDI200 antibodies to neutralize the test article's ability of inducing apoptosis. The JOM2-2199 (CL23 SCL20) cell line is used, a Jurkat Fas-deficient cell line that has been transduced with the extracellular domain of EDAR and the intracellular domain of Fas. Cells are incubated in the presence of EDI200, activating the EDAR-Fas receptor and subsequent Fas signaling cascade resulting in inhibition of proliferation and survival.

Assessment of the assay is carried out through the optimization of assay conditions, examining the test article activity curve and examining the inhibition activity curve. The impact of cell concentration and cell passage number (up to passage 15) is assessed to determine optimal assay conditions. All additional experiments are conducted using the optimal cell concentration (cell number per well) as well as the optimal range of cell passage numbers.

ELISA based assays for the detection of neutralizing antibodies may also be designed and performed. The design and testing of ELISA assays are known in the art. Briefly in this assay, the substrate is coated with hDAR-mFc followed by standard blocking Small amounts of biot-EDI200 (biotinylated EDI200) pre-incubated with serum (˜50 ng/mL) are added. If blocking or neutralizing antibodies are present, the signal will be blocked.

### Test Article Activity Curve

Two-fold serial dilutions of EDI200 in the neat negative human matrix pool are assayed in triplicate wells, as two curves per run and two independent runs for a total of four dilution curves. Curve performance is assessed by intra and inter dilutional precision which must be ≦30% CV to be acceptable. Dilutions span the detection range of the assay, aiming for an O.D. range of 3.0 to 0.2, at a minimum. A test article concentration is selected to be used for the remainder of assay validation. This concentration falls within the linear portion of the curve such that the O.D. signal is responsive to the addition of various concentrations of neutralizing antibody.

### Inhibition Activity Curve

The previously determined test article concentration is tested in the presence of various concentrations/titrations of the anti-EDI200 antibody which is used to construct an inhibition activity titration curve. Test article samples with the determined concentration are pre-incubated with the various concentrations/titrations of the anti-EDI200 antibody for 60±5 min at room temperature prior to analysis. Curve performance is assessed across 6 total curves performed as two curves per run for three independent runs across two days and two independent analysts. From the measured values across all six curves, the inter and intra dilutional precision is calculated and deemed acceptable if ≦30% CV. Five neutralizing antibody quality control (QC) levels are chosen such that they span the linear range of the curve: QC high (QCH), 3 QC mid (QCM1, QCM2 and QCM3) and QC low (QCL). Each plate also contains triplicate wells of Maximum Proliferation Control comprised of human serum pool analyzed in the absence of neutralizing antibody or EDI200, as well as a Negative Control comprised of human serum pool analyzed in the absence of neutralizing antibody but in the presence of EDI200.

The minimum required dilution (MRD) is determined to consider the minimum interference from matrix components, and to determine the minimum dilution that generates a signal approaching that to the signal of non-specific binding (NSB) of the cell culture media. The Maximum Proliferation Control and Negative Control are run in triplicate wells using cell culture media (to determine non-specific binding) and a series of pooled human serum dilutions prepared using cell culture media. The OD signal for each sample is reported and evaluated to select the assay MRD. The MRD will be implemented to all subsequent validation runs. For each sample, the OD signal % CV must be ≦30%.

### Validation Procedures

Five QC levels containing positive control antibody (QCH, QCM1, QCM2, QCM3 and QCL) comprise a QC set and are analyzed in the presence and absence of EDI200. The Maximum Proliferation Control is comprised of pooled human serum without EDI200 to achieve maximum cell proliferation and is analyzed in the absence of neutralizing antibody. The Negative Control is comprised of pooled human serum spiked with EDI200 to achieve maximum inhibition of cell proliferation and is analyzed in the absence of neutralizing antibody. Table 28 summarizes the control conditions that are included with each run, in triplicate wells. Optical Density (OD) Ratio is calculated for the QC samples as described in Table 28. For each validation run, the OD signal % CV is ≦30% for each QC level, Negative Control and Maximum Proliferation Control, and is as follows:

NC<QCL<QCM3<QCM2<QCM1<QCH<MPC.

The mean OD of the three Negative Control replicates is used for OD Ratio calculations.

TABLE 28
Control conditions

| | | | OD |
|---|---|---|---|
| Sample | Analyte | Serum | Ratio |
| Maximum Proliferation | — | Blank human serum pool | — |
| Control (MPC) | | | |
| Negative Control (NC) | EDI200 | Blank human serum pool | — |
| Positive Control/QC set | EDI200 | Human serum pool contain- | QC/NC |
| (QC) | | ing surrogate positive | |
| | | control antibody | |

### Matrix Testing

At least 20 human serum lots procured from a commercial source are tested in duplicate wells across two days. Each serum lot is run in duplicate wells under the Negative Control condition outlined in Table 28. For each run, the OD % CV is ≦30%.

### Negative Cut-Off (NCO) Determination

For this cell-based neutralizing antibody assay, the negative cut-off (NCO) point is determined experimentally and defined as the response level below which the sample is considered negative. Response values at or above the NCO point are considered positive.

The NCO is determined from at least 20 human serum lots using parametric approaches. The mean response and % CV for each sample is reported. .

### Example 10

### Phase 1, Safety and Pharmacokinetic Study of EDI200 in X-Linked Hypohidrotic Ectodermal Dysplasia (XLHED) Adults

The objective of this study was to assess the safety, tolerability, immunogenicity and pharmacokinetics of EDI200 administered to XLHED-affected adults. The exploratory objective of the study was to assess pharmacodynamic/biologic activity of EDI200 administered to XLHED-affected adults.

### Materials and Methods

### Treatment Administered

EDI200 was provided as a sterile solution for intravenous infusion in 3 ml glass vials at 5 mg/ml. All study drug supplies, including EDI200, were stored frozen at −60° C. to −90° C.

### Procedure

Six XLHED-affected adult individuals were divided into two cohorts. Subjects in cohort 1 were dosed at 3 mg/kg/dose. Subjects in cohort 2 were dosed at 10 mg/kg/dose. The dosing regimen in each cohort involved a total of 5 doses of EDI200 IV on Days 0, 4, 7, 11, and 14. Subjects were followed for a total of 6 weeks following first dose of study drug, approximately 4 weeks following last dose of study drug.

### Results

### Safety Assessment

The safety assessment variables were adverse events, concomitant medications, vital signs, weight, electrocardiogram (ECG), physical examination findings, hematology, clinical chemistry, and urinalysis laboratory test results.

Overall, 83.3% (5/6) of subjects experienced 20 treatment emergent adverse events (TEAE). Cumulatively, 16 of the 20 TEAEs were of mild intensity, 3 were moderate and one was severe. The severe TEAE was determined to be not related to study drug. There were no serious TEAEs and there were no TEAEs leading to discontinuation of study drug.

### Pharmacokinetic (PK) Parameters and Immunogenicity

Pharmacokinetic parameters included maximum plasma concentration (Cmax), area under the curve (AUC), clearance, and elimination half-life (t½) (see Table 29). A compartmental model fit the serum concentration data for EDI200 well. Variability between subjects was small. The typical value for clearance was 21.4507 L/day. Mean AUC and Cmax values are markedly smaller than values reported for no adverse effect level doses in preclinical studies.

TABLE 29
AUC and Cmax Values

| | Cumulative | | Cumulative | Cmax, | Cmax, |
|---|---|---|---|---|---|
| | Dose | AUC/dose | AUC | Dose 1 | Dose 5 |
| | (mg) | (ng/ml · hours) | (ng/ml · hours) | (ng/ml) | (ng/ml) |
| Mean | 2715.25 | 604512 | 3022562 | 68762 | 59116 |
| SD | 1709.431 | 318678 | 1593390 | 50957 | 39133 |

Immunogenicity was measured by the presence or absence of anti-EDI200 antibodies in the serum. There was no correlation of antibody titer with EDI200 dose or clinical events.

### Pharmacodynamic (PD)/Biologic Activity

The pharmacodynamic (PD)/biologic activity assessment variables included hair number and growth properties, pulmonary function and exhaled nitric oxide (eNO) levels, sweat duct density, sweat rate, saliva quantitation, tearing and dry eye evaluation, and skin biopsy for expression profile.

Hair number and growth properties were analyzed on phototrichograms obtained from the 4 male study subjects, 2 from each cohort. In an exploratory statistical analysis of the data from the 2 subjects from the 10 mg/kg/dose cohort, a statistically-significant 50% increase in hair growth rate from pre- to post-dosing was found.

Pulmonary function (forced vital capacity and forced expiratory flow over 1 second) and eNO levels at D42 compared to baseline for the XLHED male and female population was not statistically significant. However, over the course of the study all 3 subjects in cohort 2 receiving the higher dose of EDI200 had a decrease in eNO levels, considered a measure of pulmonary inflammation.

No statistically significant changes were observed in males from either dosing cohort for sweat duct density or sweat rate. No statistically significant changes were observed among individual treatment groups for saliva quantitation. No statistically significant changes were observed among cohorts for tear stability and tear production. However, in the cohort 2 receiving the higher dose of EDI200, 5 of 6 eyes examined showed an improvement in tear production from baseline to D42.

Ocular surface disease index (OSDI) change from baseline to D42 did not show improvement in cohort 1. However, in cohort 2, both male subjects showed an improvement in ocular surface disease index score from baseline to D42.

Four mm diameter punch biopsies of skin were obtained from the forearm of male subjects. Ribonucleic Acid (RNA) isolated from the skin biopsies was assayed in molecular expression analysis. Results from pre-dosing and post-dosing samples were compared for evidence of an EDI200 biologic response pattern in XLHED-affected adults. Results are pending.

### Conclusion

The Phase 1 study of EDI200 administered to XLHED-affected men and women successfully met its goals of enrolling two cohorts and completing a course of 5 IV doses in each of the 6 subjects. The primary objectives of demonstrating safety and PK were met. No clinical events or significant changes in PK were assessed as affected by the presence of EDI200 neutralizing antibodies. Statistical evaluation of EDI200 bioactivity/PD endpoints may be of limited value due to the small sample size of individuals in the study and low EDI200 doses not powered for efficacy.

### Example 11

### Phase 2 Dose-Escalation Safety, Pharmacokinetics, Immunogenicity and Pharmacodynamics/Efficacy Study of EDI200 in Male Infants with X-Linked Hypohidrotic Ectodermal Dysplasia (XLHED)

The primary objective of this study is to assess the safety, pharmacokinetics and immunogenicity of EDI200 administered to XLHED-affected neonates. The pharmacodynamic/efficacy objectives of this study are to assess the pharmacodynamics/efficacy of EDI200 administered to XLHED-affected neonates and compare clinical data and medical history obtained from untreated male siblings to that of the XLHED-affected neonates receiving EDI200.

### Materials and Methods

### Treatment Administered

EDI200 is provided as a sterile solution for intravenous infusion. All study drug supplies, including EDI200, are stored frozen at −60° C. to −90° C.

### Procedure

Six to ten XLHED-affected neonate individuals between 2 and 14 days old are divided into two cohorts. Subjects in cohort 1 are dosed at 3 mg/kg/dose. Subjects in cohort 2 are dosed at 10 mg/kg/dose. The dosing regimen in each cohort is a total of 5 doses of EDI200 IV on Days 0, 4, 7, 11, and 14. Subjects are followed for 1 week following the last dose of study drug, and subsequently at 2 months, 4 months, and 6 months following the last dose of EDI200.

### Results

### Study Evaluations

Primary outcome measures for all subjects will be safety, PK and immunogenicity. Study duration is 6 months with all subjects rolling over into a long-term extension study providing yearly evaluations. Pharmacodynamic/efficacy objectives in the Phase 2 neonate study will be limited by the timeline for ectodermal development that often exceeds 6 months, e.g. dentition. Therefore, several of these endpoints will be incorporated into the extension study protocol. There will be assessment of the following: (1) endpoints relevant to the common clinical findings in XLHED using age-appropriate technologies, e.g. growth and development, infections and hospitalizations, sweat duct counts and stimulated sweat production, pre-treatment dentition, and thermoregulation; (2) change from baseline in craniofacial structures using a non-invasive facial recognition software program based on subject digital facial photographs (Appendix 1); and (3) change in molecular expression profile using skin biopsy samples obtained pre- and post-study drug exposure.

### Example 12

### Development and Validation of a Computer Assisted Screening Technology to Identify Asymptomatic XLHED Candidates for EDI200 Intervention

Development of a computer assisted screening method to measure and identify asymptomatic XLHED affected subjects may be carried out using known methods in the art. A screening process is performed whereby a facial image of a subject, (e.g., neonate, youth or adult) is analyzed to identify XLHED affected subjects that exhibit characteristic asymptomatic phenotypes from birth. Relative facial measurements and proportions are taken and synthesized into a diagnostic score relative to known affected individuals. Once developed, such an assay may undergo feasibility testing and/or further development. The method is then validated for use to identify human neonates for EDI200 intervention in a similar manner as the non-human primate methods taught herein.

1. A pharmaceutical composition comprising at least one protein monomer, said protein monomer comprising SEQ ID NO: 1 and wherein said at least one protein monomer is glycosylated at positions Asn76 and Asn302 and a pharmaceutically acceptable excipient.

2. The pharmaceutical composition of claim 1, in unit dosage form.

3. The pharmaceutical composition of claim 2, wherein at least three protein monomers form said unit dosage form.

4. The pharmaceutical composition of claim 2, wherein at least six protein monomers form a hexamer complex in said unit dosage form.

5. The pharmaceutical composition of claim 2, wherein six protein monomers form a hexamer complex in said unit dosage form.

6. The pharmaceutical composition of claim 1, wherein the pharmaceutically acceptable excipient is a diluent comprising sodium phosphate and sodium chloride.

7. The pharmaceutical composition of claim 6, further comprising one or more surfactants and/or detergents.

8. The pharmaceutical composition of claim 7, further comprising polysorbate 20.

9. The pharmaceutical composition of claim 5, comprising about 0.5% of the hexamer complex, about 20 mM sodium phosphate, about 300 mM sodium chloride and about 0.02% polysorbate 20 by volume.

10. The pharmaceutical composition of claim 9, having a volume of 2.1 mL.

11. The pharmaceutical composition of claim 2, wherein the unit dose is from about 0.10 mg/kg to about 200 mg/kg.

12. The pharmaceutical composition of claim 11, wherein unit dose is selected from the group consisting of from about 0.15 mg/kg to about 1.5 mg/kg, from about 0.3 mg/kg to about 3 mg/kg, from about 1 mg/kg to about 15 mg/kg, and from about 10 mg/kg to about 30 mg/kg.

13. A method for correcting, altering or mitigating one or more phenotypic presentations of ectodermal dysplasia in a human diagnosed with or suspected of having ectodermal dysplasia comprising, administering to said human pharmaceutical composition according to claim 1.

14. The method of claim 13, wherein the pharmaceutical composition is administered in unit dosage form.

15. The method of claim 14, where the unit dose is administered by intravenous injection using continuous infusion wherein the infusion rate is selected from the group consisting of from about 0.5 ml/kg/hour to about 5 ml/kg/hour, from about 1.5 ml/kg/hour to about 10 ml/kg/hour or from about 3 ml/kg/hour to about 20 ml/kg/hour.

16. The method of claim 15, where the unit dose is administered by intravenous injection using continuous infusion wherein the infusion rate is about 5 ml/kg/hour.

17. The method of claim 15, wherein the continuous infusion occurs over a period of time from about 30 min to about 5 hours.

18. The method of claim 17, wherein the period of time is approximately 2 hours.

19. The method of claim 15, wherein administration occurs at between 15°-25° Celsius.

20. The method of claim 13, wherein the phenotypic presentation of ectodermal dysplasia is selected from the group consisting of: missing teeth, abnormally shaped teeth, abnormal morphology or lack (or reduced number) of sweat glands, lack of Meibomian glands, lack of glands of the upper respiratory tract, lack of sebaceous glands, lack of salivary glands, lack or abnormal morphology of various types of hair, and alopecia.

21. The method of claim 13, wherein the ectodermal dysplasia is X-linked hypohidrotic ectodermal dysplasia (XLHED).

22. A method of treating a subject having XLHED, comprising contacting said subject with the pharmaceutical compositions of claim 1.


SEQUENCE LISTING (8 sequences)

SEQ ID NO: 1 - protein, 380 aa, Artificial sequence
  EDI200 monomer
KTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKADKAGTRENQPAVVHLQGQGSAIQVKNDLSGGVLNDWSRITMNPKVFKLHPRSGELEVLVDGTYFIYSQVEVYYINFTDFASYEVVVDEKPFLQCTRSIETGKTNYNTCYTAGVCLLKARQKIAVKMVHADISINMSKHTTFFGAIRLGEAPAS

SEQ ID NO: 2 - DNA, 18 bp, Artificial sequence
  PCR Primer
atttaggtgacactatag

SEQ ID NO: 3 - DNA, 36 bp, Artificial sequence
  PCR Primer
tccagtgtggtggaattcatggctatcatctacctc

SEQ ID NO: 4 - DNA, 20 bp, Artificial sequence
  Forward Primer
taatacgactcactataggg

SEQ ID NO: 5 - DNA, 18 bp, Artificial sequence
  Forward Primer
ccgacggctccttcttcc

SEQ ID NO: 6 - DNA, 18 bp, Artificial sequence
  Reverse Primer
ggaagaaggagccgtcgg

SEQ ID NO: 7 - DNA, 18 bp, Artificial sequence
  Reverse Primer
aggcacagtcgaggctga

SEQ ID NO: 8 - DNA, 7336 bp, Artificial sequence
  P449 Plasmid
aatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactaggcttttgcaaaaagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattagcttggtactaatacgactcactatagggagacccaagctggctaggtaagcttggtaccgagctcggatccactagtccagtgtggtggaattcatggctatcatctacctcatcctcctgttcaccgctgtgcggggcaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaagctgataaagctggaactcgagaaaaccagccagctgtggtgcatctacagggccaagggtcagcaattcaagtcaagaatgatctttcaggtggagtgctcaatgactggtctcgcatcactatgaaccccaaggtgtttaagctacatccccgcagcggggagctggaggtactggtggacggcacctacttcatctatagtcaggtagaagtatactacatcaacttcactgactttgccagctatgaggtggtggtggatgagaagcccttcctgcagtgcacacgcagcatcgagacgggcaagaccaactacaacacttgctataccgcaggcgtctgcctcctcaaggcccggcagaagatcgccgtcaagatggtgcacgctgacatctccatcaacatgagcaagcacaccacgttctttggggccatcaggctgggtgaagcccctgcatcctagcggccgctcgagtctagagggcccgcggttcgaacaaaaactcatctcagaagaggatctgaatatgcataccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttc